(19) United States
(12) Patent Application Publication
(10) Pub. No.: US 2003/0032809 A1
(43) Pub. Date: Feb. 13, 2003

(54) Optimized procedures for the manufacture of paroxetine salts

(76) Inventors: Subhash P. Upadhyaya, Chicago, IL (US); Bruce Ronsen, River Forest, IL (US); Bryant Wojcik, LaGrange, IL (US)

Correspondence Address: Bruce D. Grant, Morrison & Foerster LLP, Suite 500, 3811 Valley Centre Drive, San Diego, CA, 92130, US

(21) Appl. No.: 10/133,726

(22) Filed: Apr. 25, 2002

Related U.S. Application Data
(60) Provisional application No. 60/333,530, filed on Nov. 20, 2001.
(60) Provisional application No. 60/295,471, filed on Jun. 1, 2001.
(60) Provisional application No. 60/290,411, filed on May 11, 2001.
(60) Provisional application No. 60/286,590, filed on Apr. 25, 2001.

(51) Int. Cl. C 07D 4 7/02

(52) U.S. Cl. 546/197

(57) ABSTRACT

Described herein are methods for manufacturing amorphous paroxetine hydrochloride by mixing a carboxylic acid salt of paroxetine with hydrogen chloride and isolating the amorphous paroxetine hydrochloride. Also described herein are methods for manufacturing substantially pure paroxetine free base and methods for obtaining paroxetine salts such as paroxetine acetate, paroxetine trifluoroacetate, and paroxetine formate. Also described are methods for preparing paroxetine carbonate, paroxetine phosphate, paroxetine sulfate, and analogous salts thereof. The paroxetine salts may be formulated for the treatment of medical disorders as described.

### CROSS-REFERENCE TO RELATED APPLICATIONS

This application claims the benefit of the Apr. 25, 2001 filing date of U.S. application Ser. No. 60/286,590, the May 11, 2001 filing date of U.S. application Ser. No. 60/290,411, and the Jun. 1, 2001 filing date of U.S. application Ser. No. 60/295,471, each of which is entitled “Paroxetine Salts And Processes For The Manufacture Of Paroxetine Salts” and names S. Upadhyaya and B. Ronsen as inventors. This application also claims the benefit of the Nov. 20, 2001 filing date for U.S. application Ser. No. 60/333,530, which is entitled “Optimized Procedures for the Manufacture of Paroxetine Salts” and names S. Upadhyaya and B. Ronsen as inventors. Each of these applications is incorporated herein by reference in its entirety.

### TECHNICAL FIELD

The invention relates to a class of compounds known as paroxetines. In particular, disclosed herein are processes for purifying paroxetine free base and preparing paroxetine salts.

### BACKGROUND

The compound paroxetine, (−)-trans-4R-(4′-fluorophenyl)-3S-[(3′,4′-methylenedioxyphenyl)oxymethyl]piperidine has been known for quarter of a century and has been used for the treatment of depression, premature ejaculation, social anxiety disorder, stress, and other central nervous system conditions. The chemical structure of paroxetine (1) is set forth below.

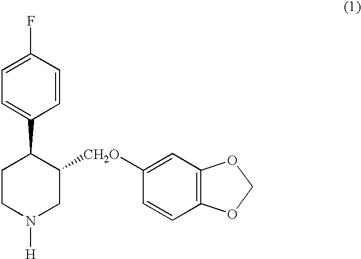

Processes for manufacturing substantially pure paroxetine salts are required for producing pharmaceuticals for treating these medical conditions.

Christensen et al. described the synthesis of paroxetine free base and converted it to maleate salt for use in biological and pharmacological screenings in U.S. Pat. No. 4,007,196. Subsequently, most of such screenings of paroxetine were carried out using paroxetine acetate because of its excellent water solubility profile and therapeutic acceptability. Psychopharmacology 57: 151-153 (1978), Psychopharmacology 68: 229-233 (1980), and Eur. J. Pharm. 47: 351-385 (1978). However, due to the lack of well defined manufacturing process having good scale-up potential, reproducibility, and acceptable yield of a substantially pure paroxetine acetate, a number of other salts were utilized as acceptable salts of therapeutic agents. For example, Barnes et al. disclosed a process for making paroxetine hydrochloride hemihydrate in U.S. Pat. No. 4,721,723. Also, several forms of paroxetine hydrochloride anhydrate are known, as discussed for example in U.S. Pat. No. 5,856,493. However, these paroxetine salts have undesirable physico-chemical properties as evidenced by their low water solubility. Moreover, crystallization methods used to obtain these paroxetine salts were low yielding and produced mostly solvated material.

In U.S. Pat. No. 4,721,723, Barnes et al. discussed methods for preparing paroxetine acetate and paroxetine hydrochloride. In U.S. Pat. No. 5,874,447, Bennecker et al. discussed methods for preparing paroxetine acetate and paroxetine methane sulfonate. U.S. Pat. No. 4,721,723 discussed a process in which 1-chloroethyl chloroformate or vinyl chloroformate were utilized to produce a 1-chloroethyl carbamate derivative in the presence or absence of a base known as a proton sponge. The carbamate derivative was then, without removal of the proton sponge, hydrolyzed in situ in boiling methanol to produce crude paroxetine hydrochloride. Paroxetine acetate was formed in aqueous solution from the crude free base, from which crystalline paroxetine hydrochloride hemihydrate was precipitated directly by addition of hydrochloric acid. Paroxetine hydrochloride hemihydrate was also obtained from the relatively impure paroxetine starting material by seeding. Paroxetine hydrochloride was mixed with charcoal and alumina to purify some impurities away from paroxetine before crystallization.

However, the methods discussed in U.S. Pat. No. 4,721,723 and U.S. Pat. No. 5,874,447 are low yielding or fail to produce paroxetine acetate with acceptable purity. Thus, these methods have poor scale-up potential. Moreover, replacing a stronger acid such as methane sulfonic acid by a weaker acid like acetic acid is not a viable process.

Other methods have been described for the synthesis of paroxetine free base. For example, Christensen et al. in U.S. Pat. No 4,007,196 and Wang et al. in European Patent Application No. 0810225A1, described processes for reacting N-methylparoxetine with phenyl chloroformate or alkyl chloroformate to produce a carbamate intermediate, the latter of which was then hydrolyzed in presence of a strong base in boiling solvent. These processes did not address the problem of impurities in the paroxetine free base end product, which inevitably lowered purity and yield of paroxetine acid salt synthesized from the base, thereby making the process inefficient and non-scalable.

Thus, there is a need for efficient processes that produce highly pure paroxetine free base and highly pure paroxetine salts.

### SUMMARY

The need for substantially pure paroxetine salts is satisfied by this invention. Featured herein is a process for preparing amorphous paroxetine hydrochloride which comprises mixing a carboxylic acid salt of paroxetine, such as paroxetine acetate and paroxetine formate, with hydrogen chloride and isolating the resulting amorphous paroxetine hydrochloride. Also featured herein is a process for purifying paroxetine free base from which paroxetine salts may be synthesized. Embodiments of the invention include methods for preparing paroxetine salts such as paroxetine acetate, paroxetine trifluoroacetate, and paroxetine formate. Also provided are methods for preparing paroxetine salts such as paroxetine carbonate, paroxetine phosphate, paroxetine sulfate, and hydrogenated salts thereof. The paroxetine salts may be formulated for treatment of medical disorders as described herein, or can be utilized for the manufacture of other paroxetine salts.

### BRIEF DESCRIPTION OF THE FIGURES

FIG. 1 depicts generalized procedures for utilizing paroxetine free base and paroxetine salts in processes for preparing paroxetine salts.

FIGS. 2A and 2B depict an FT-IR spectrum and an X-ray diffractogram, respectively, for paroxetine formate synthesized according to Example 6.

FIGS. 3A and 3B depict an FT-IR spectrum and an X-ray diffractogram, respectively, for paroxetine acetate synthesized according to Example 8.

FIG. 4 depicts X-ray powder diffraction data for amorphous paroxetine hydrochloride synthesized according to Example 12, which was obtained by a Schimadzo XRD-6000 instrument.

FIG. 5 depicts X-ray powder diffraction data for amorphous paroxetine hydrochloride synthesized according to Example 13.

FIG. 6A is a diagram of a Buchi rotary evaporator model R-153 and vacuum pumps discussed in Example 15, including (a) Buchi vacuum pump model V-512 with vacuum controller B-712, (b) Welch vacuum pump Dirrectorr 8910, (c) 3-Way TFE vacuum valve, (d) Dewar with liquid nitrogen and vacuum trap, (e) Rotary evaporator condenser, (f) 10 L receiver, (g) 500 ml receiver, (h) 20 L flask, (i) water bath, and (j) Dewar flask filed with liquid nitrogen.

FIG. 6B depicts X-ray powder diffraction data for amorphous paroxetine hydrochloride synthesized according to Example 15.

FIG. 7 depicts X-ray powder diffraction data for the paroxetine hydrochloride salt synthesized according to Example 16.

### MODES OF CARRYING OUT THE INVENTION

Processes for Purifying Paroxetine Free Base

Purified paroxetine free base can be used for the synthesis of paroxetine salts. Crude or impure paroxetine free base may be purified by a process which comprises mixing the impure paroxetine free base with a water immiscible organic solvent, water, and an acid to form a water soluble paroxetine salt that corresponds to the acid. An example of a suitable acid is a carboxylic acid, including acetic acid, trifluoroacetic acid, formic acid, and propionic acid. The paroxetine salt thus formed is soluble in the aqueous phase and impurities may be removed by discarding the organic phase using separation techniques well-known in the art and described herein. The aqueous phase may be mixed with a water immiscible organic solvent and an aqueous solution of a base, and impurities present in a scum located between the aqueous layer and organic layer and inorganic layer may be removed by a suitable separation technique known in the art, for example, by filtration. Although the scum does not typically contain a large amount of impurities, removal of those impurities can significantly improve crystallization of paroxetine salts synthesized from the purified paroxetine free base (see e.g. Example 10). After the aqueous phase is separated from the water immiscible organic solvent by separation techniques known in the art and described herein, the resulting paroxetine free base in the water immiscible organic solvent is often substantially pure.

As used herein, the term “purifying” refers to a process of reducing the concentration of non-paroxetine components (e.g., not paroxetine free base or paroxetine salt) in a mixture comprising paroxetine free base or a paroxetine salt. As used herein, the term “substantially pure” refers to paroxetine free base or a paroxetine salt greater than 90% or 95% free from impurities, more often greater than 96%, 97%, or 98% free from impurities, and normally greater than 99% free from impurities. The terms “crude” or “impure” as used herein refer to paroxetine free base that is less than 90% free from impurities, less than 70%, less than 60%, or less than 50% free from impurities.

Any water immiscible organic solvent in which paroxetine free base dissolves may be used. Examples of water immiscible organic solvents include toluene, benzene, xylenes, chloroform, methylene chloride, acetonitrile, ketones (e.g., dimethylketone, methylethylketone, and diethylketone), dimethylformamide, dimethylsulfoxide, esters (e.g., ethyl acetate), ethers (e.g., diethylether and dipropylether), 1,4-dioxane, tetrahydrofuran, pentanes, hexanes, heptanes, trichloroethene, or suitable mixtures of thereof. An example of a non-aqueous solvent is toluene, which may be used in an amount of about 5 ml to about 12 ml per gram of paroxetine base or paroxetine salt.

A base capable of converting a water soluble paroxetine salt into paroxetine free base may be used in this process. Examples of bases are KOH or NaOH. Also, an acid that converts paroxetine free base into a water soluble paroxetine salt may be used in this process. Examples of acids are acetic acid, formic acid, and propionic acid, and any other carboxylic acid which can be used to prepare the water soluble paroxetine salt from paroxetine base. Typically, about a stoichiometric amount of acid is utilized for the conversion of paroxetine base into paroxetine salt.

The process may be carried out using an impure water soluble paroxetine salt as a starting material instead of using paroxetine free base as a starting material. When a water soluble paroxetine salt is used as a starting material, it is often extracted with a water immiscible organic solvent to remove impurities in the organic phase.

The content of purified paroxetine free base can be estimated with approximately 95% certainty by withdrawing an aliquot of the solution, evaporating solvent from the aliquot to yield paroxetine free base as an oil that is substantially free of solvent, and calculating the amount of free base in the solution from the amount of paroxetine free base obtained as the oil in the aliquot. For example, 50 ml of a purified solution containing paroxetine free base may be evaporated to dryness under reduced pressure in a water bath having a temperature of about 30° C. to 60° C.

Water may be removed from the purified paroxetine free base. For example, water may be removed by contacting the solution with a drying agent and then filtering away the drying agent. Examples of drying agents are anhydrous magnesium and sodium sulfate. Also, water may be removed by evaporation or distillation. For example, distilling approximately 10 to 20 percent of the water immiscible solvent under reduced pressure may significantly reduce water content.

Processes for Preparing Paroxetine Salts

Paroxetine salts may be prepared from purified paroxetine free base, where paroxetine free base is mixed with a solution comprising an acid to yield the corresponding salt of the acid, and the paroxetine salt may be isolated. Solid paroxetine salt may be washed and solvent may be removed.

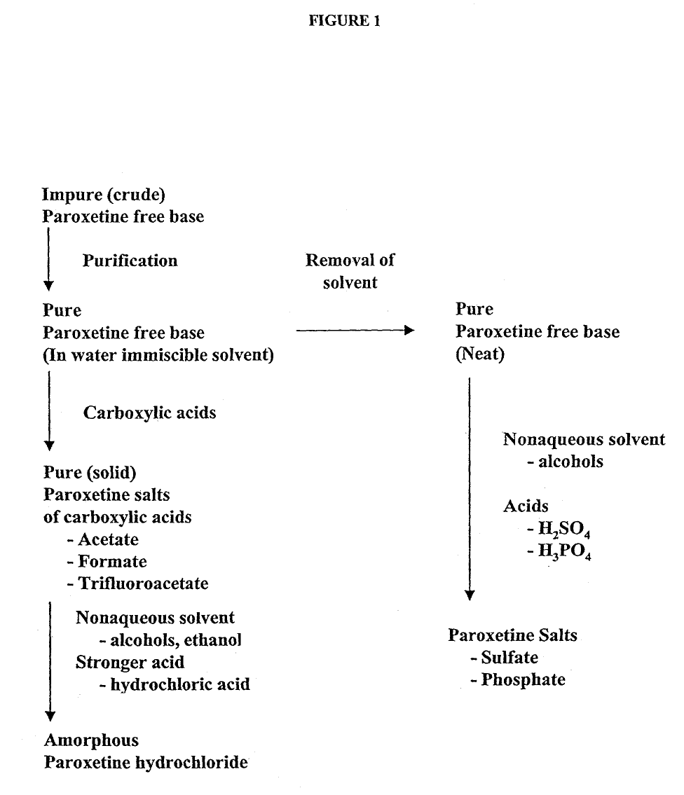

Acid moieties may be introduced to paroxetine base by various means to yield pharmaceutically acceptable salts (see for example FIG. 1). In a fusion method, the acidic moiety may be introduced in neat form. In a solution method, the acidic moiety may be introduced in neat form or by a solvent, such as a non-aqueous solvent, which may be removed later.

Generally, paroxetine salts may be prepared by reacting paroxetine free base with an approximate stoichiometric amount of the desired salt-forming inorganic or organic acid. The content of paroxetine base in solution is typically estimated with 95% certainty, and less than a stoichiometric amount of the salt-forming acid is often used in the process, as described herein. For example, up to about 0.95 molar equivalents of the acid is sometimes utilized and about a molar equivalent of the acid is often utilized.

A solvent described herein may be included in the solution comprising the acid. For example, water immiscible organic solvents described herein as well as non-aqueous solvents may be utilized. Examples of non-aqueous solvents are alcohols, such as methanol, ethanol, propanol, butanol, pentanol, hexanol, heptanol, octanol, and branched chain alcohols (e.g., isopropanol and isobutanol). For example, toluene may be used when preparing paroxetine acetate and paroxetine formate and an alcohol such as ethanol may be used when preparing paroxetine hydrochloride, paroxetine sulfate, and paroxetine phosphate. A total volume of solution to paroxetine base ratio of 7.5 ml to 10 ml of solution per gram of paroxetine base is normally utilized, although other solvent ratios may be used.

Solid paroxetine salt may be isolated using methods known in the art and described herein. For example, the salt may be isolated by crystallization and filtration, as discussed herein, where the salt may be washed and air dried. Also, the salt may be present as a solid after paroxetine base is treated with an acid without carrying out separate crystallization procedures, in which case, the solid may be filtered and washed.

Pharmaceutically acceptable paroxetine salts may be obtained from organic or inorganic acids. Lists of suitable salts are found in Remington's Pharmaceutical Sciences, 17th ed., Mack Publishing Company, Easton, Pa., 1985, p. 1418, the disclosure of which is hereby incorporated by reference. Salts derived from inorganic acids include hydrochloric, hydrobromic, sulfuric, sulfamic, phosphoric, nitric, carbonic and like acids. Salts prepared from organic acids include formic, acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethanesulfonic, ethanedisulfonic, oxalic, isethionic, and like acids.

An example of a paroxetine salt is paroxetine acetate. Substantially pure paroxetine free base can be mixed with acetic acid to form paroxetine acetate substantially free of bound solvent, as described herein. As noted above, a substantially anhydrous solution of paroxetine free base is suitable for synthesis of paroxetine acetate from paroxetine free base. Water in the solution of free base lowers yields of paroxetine acetate since paroxetine acetate is highly soluble in water. Other examples of paroxetine salts are paroxetine trifluoroacetate and paroxetine formate. Other salts include paroxetine carbonate, paroxetine sulfate, paroxetine phosphate, and hydrogenated salts thereof. For example, mixing paroxetine free base with carbonic acid will yield paroxetine carbonate and/or paroxetine hydrogen carbonate; mixing paroxetine free base with sulfuric acid will yield paroxetine sulfate and/or paroxetine hydrogen sulfate; and mixing paroxetine free base with phosphoric acid will yield paroxetine phosphate, paroxetine hydrogen phosphate and/or paroxetine dihydrogen phosphate. The number of hydrogens on the paroxetine salt may be controlled by the molar ratio of paroxetine to acid utilized. For example, mixing two moles of paroxetine to one mole of carbonic acid will substantially yield paroxetine carbonate, whereas mixing one mole of paroxetine to one mole of sulfuric acid will substantially yield paroxetine hydrogen sulfate.

Paroxetine salts may be characterized in a number of manners. For example, paroxetine salts may be characterized by a melting point. Specifically, paroxetine formate can be characterized as having a melting point that is less than or equal to 101° C., paroxetine acetate can be characterized as having a melting point that is less than or equal to 125° C., paroxetine trifluoroacetate can be characterized as having a melting point that is less than or equal to 117° C., paroxetine sulfate can be characterized as having a melting point that is less than or equal to 245° C., and paroxetine dihydrogen phosphate can be characterized as having a melting point that is less than or equal to 208° C. Amorphous paroxetine hydrochloride can be characterized as having a broad melting range where the first melting occurs at less than or equal to 100° C. to produce a transparent gel which is followed by a second melting of the gel at a higher temperature and where the difference between two melting points is 10° C. or more. Also, paroxetine salts may be characterized by infrared (IR), Fourier transform infrared (FT-IR), X-ray powder diffraction, and nuclear magnetic resonance spectral characteristics, as well as by elemental analysis characteristics and optical rotation.

Paroxetine free base purified as described herein provides nearly quantitative yields of paroxetine salts. Yields greater than 60% and 70%, and often yields greater than 75%, greater than 80%, greater than 85%, greater than 90%, and sometimes greater than 95% can be obtained using the processes disclosed herein. For example, paroxetine acetate may be obtained with about an 80% yield when 1 gram of purified paroxetine free base and 0.95 equivalents of acetic acid are mixed in a total volume of 7.5 ml to 10.0 ml for final crystallization. Weight equivalents are based on the content of paroxetine free base in the solution. Solid paroxetine acetate prepared by the methods disclosed herein often has a melting point from 123° C. to 126° C., and typically a melting point from 124° C. to 125° C. Purified paroxetine acetate normally has a solubility of at least 1000 mg per one ml of purified water at room temperature.

Paroxetine salts may also be derived from other paroxetine salts. Salts of paroxetine derived from a weak acid, such as a carboxylic acid, may be utilized to prepare a second salt of paroxetine derived from a stronger acid, such as hydrogen chloride. For example, amorphous paroxetine hydrochloride may be prepared by contacting a carboxylic acid salt of paroxetine (e.g., paroxetine acetate, paroxetine trifluoroacetate, and paroxetine formate) with hydrogen chloride gas, or by mixing a carboxylic acid salt of paroxetine in a solution containing hydrochloric acid and a non-aqueous solvent such as ethanol. In the latter strategy, the solution can be mixed at elevated temperatures followed by evaporation of solvent under reduced pressure at elevated temperatures. When a carboxylic acid salt of paroxetine is mixed with an alcohol containing at least a stoichiometric amount of hydrochloric acid, an equivalent amount of carboxylic acid is normally released instantaneously, due to its weak dissociation property, to produce a clear solution of paroxetine hydrochloride.

Temperatures between about 20° C. and 40° C. are often utilized during addition of the paroxetine salt to the acid, although any temperatures in the range of 0° C. to 60° C. may be used. An amount of ethanol is normally utilized which yields a 10% to 15% by weight solution of paroxetine hydrochloride at ambient temperatures (e.g., about 20° C. to 40° C.), although other combinations of the amount of alcohol and temperatures which maintain the salt in solution may be used.

Sometimes about a molar equivalent of acid to paroxetine salt is utilized and often greater than a molar equivalent of acid to paroxetine salt is used. When greater than a molar equivalent of acid is utilized, the molar ratio of acid to paroxetine salt is sometimes between 1.0 to 4.0 and often between 1.01 to 1.1. When more than a stoichiometric amount of acid is utilized for the preparation of a paroxetine salt from a carboxylic acid salt of paroxetine, carboxylic acid released from the salt typically reacts with the alcohol in the presence of a catalytic amount of the acid to produce an ester corresponding to the carboxylic acid and the chosen solvent alcohol.

For example, since more than one mole equivalent of hydrogen chloride is often utilized for the formation of paroxetine hydrochloride, the additional hydrogen chloride normally catalyzes esterification. The rate of esterification normally depends on the amount of reactant, amount of catalyst, and reaction temperatures, and a large amount of alcohol utilized in the process may increase the rate of esterification. Although large excess of hydrogen chloride may speed esterification, only a catalytic amount of hydrogen chloride is required. Excess hydrogen chloride, if used, may not be proportionately removed from the mixture during distillation and/or evaporation, and therefore may end up in the final non-crystalline product which may affect the long-term stability and acidity of the product and impact the surrounding environment due to its corrosive nature. Since the released carboxylic acid typically reacts faster with the alcohol in the presence of a catalytic amount of hydrogen chloride at elevated temperatures, the solution is often mixed with heating in the range of about 14° C. to about 75° C. and sometimes about 45° C. to about 75° C., for a period of time to convert released carboxylic acid to ester.

In an embodiment, addition of paroxetine acetate to ethanol containing 1.06 mole equivalent of hydrochloric acid often yields a clear solution which is mixed with heating at about 60° C. for at least 60 minutes to convert released acetic acid to ethyl acetate. 1.0 mole equivalent of acid may be utilized for the formation of paroxetine hydrochloride and an additional 0.06 mole equivalent may be utilized as a catalyst for the conversion of acetic acid to ethyl acetate. Any amount of hydrochloric acid that is more than 1.06 mole equivalent may be used, and 1.1 mole equivalent or less is often utilized.

Paroxetine salts described herein may be substantially free of solvent. By “substantially free of solvent,” it is meant that the solid contains less than 20% by weight of residual solvent, often less than 10%, less then 5%, and typically less than 1%. By “substantially free of water” or “substantially anhydrous,” it is meant that the solid contains less than 20% by weight of residual water, often less than 10% and less then 5%, and sometimes less then 1%.

Any method of removing solvent that renders a homogeneous solid state dispersion or oil of a paroxetine salt may be utilized. Examples of methods for removing solvent include evaporation, which includes distillation, evaporation (e.g., vacuum evaporation, rotary evaporation, static vacuum drying, and combinations thereof), and oven drying at ambient or reduced pressure. Rotary evaporation can be utilized to obtain a paroxetine salt as described in Example 15. Evaporation of a non-aqueous solvent often renders a solid state dispersion which is homogeneous and substantially free of solvent.

It is understood that one skilled in the art of pharmaceutical formulations can determine a reasonable temperature and pressure at which the non-aqueous solvent can be removed, provided the temperature is not so high as to cause degradation or decomposition of the materials. For example, evaporation often occurs at about 20° C. to about 90° C. Vacuum evaporation may be carried out at a temperature between about 20° C. to about 80° C., sometimes about 30° C. to about 80° C. or about 35° C. to about 75° C., and often about 60° C., and at a pressure sometimes between 10 Torr and 300 Torr and often between about 1 Torr to about 100 Torr. The temperature may be held constant or nearly constant throughout a vacuum evaporation procedure. Different pressures may be utilized in stages in a vacuum evaporation procedure (e.g., two, three, four, or more stages).

For example, vacuum evaporation of a clear solution of paroxetine hydrochloride in ethanol may be carried out in three stages in a rotary evaporator. In the first stage, the bulk of the solvent may be distilled by slowly decreasing pressure to about 60 Torr to produce a concentrated solution of paroxetine hydrochloride as a thick transparent oil. House vacuum or a diaphragm pump with a pressure controller may be utilized. In the second stage, pressure may be reduced to about 1 Torr to about 10 Torr by a diaphragm pump, for example. Also, pressure may be reduced by engaging a more powerful pump capable of quickly, for example, within two minutes, lowering pressure in an empty system to about 1 Torr or lower. Direct drive or belt drive rotary-vane oil ring pumps are such high vacuum pumps and may be utilized for this purpose. As the pressure decreases, more solvent may be distilled and a foam is typically produced at about 20 Torr or about 10 Torr or lower, depending on the temperature and thickness of the oil. In the third stage, the foam may be dried further in a rotary evaporator at about 10 Torr to about 1 Torr pressure for about one hour or more. Also, pressure may fluctuate within a specific stage. The temperature may be relatively constant throughout all of the stages, or the temperature may spontaneously vary or be intentionally modified at different stages. Also, the period of time for one stage may be the same or different as the period of time for another stage. In addition to a high vacuum pump, the system may also require an efficient cold trap before the high vacuum pump for cooling of the solvent collected in the receiver as illustrated in Example 15; this arrangement can efficiently remove solvent vapors from the system and thus lower pressure to about 1 Torr for sufficiently foaming the paroxetine hydrochloride oil.

Evaporation may yield the paroxetine salt in the form of an oil or a foam, either of which may be stored, shipped, or be subjected to further processing. For example, the oil may be collected and processed to remove solvent, which may carried out by evaporating the solvent at a temperature between about 35° C. and about 75° C. and often about 60° C., and at a pressure between 60 Torr to 0.01 Torr, often between about 30 Torr and about 0.1 Torr. The oil may be subjected in a rotary evaporator to a pressure in the range of about 0.01 Torr to about 50 Torr, and sometimes from about 0.1 Torr to about 10 Torr, for the production of foam. The foam is typically amorphous and may be collected and dried to yield substantially pure solid paroxetine salt in amorphous form.

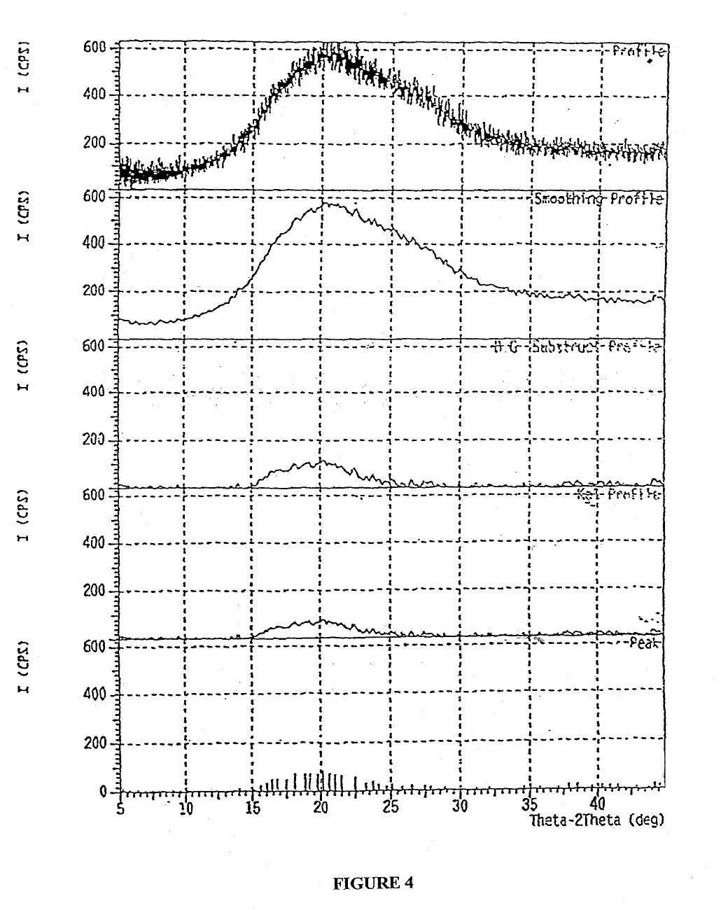

Evaporation may also yield the paroxetine salt in the form of an amorphous solid. For example, the methods described herein can yield amorphous solid paroxetine hydrochloride in which no non-amorphous material is detected. By “amorphous,” it is meant that no crystalline or ordered solid can be detected within the dried paroxetine salt. The degree to which a solid is amorphous can be readily determined as amorphous solids have certain spectral characteristics. For example, amorphous solids typically do not exhibit a definite melting temperature and instead transition from a solid form to a liquid form over a relatively large temperature range, as described herein. Also, amorphous solids often do not elicit definite spectral signals when samples are tested using powdered X-ray diffraction analysis, and instead, exhibit a halo as shown in FIGS. 4, 5, 6B, and 7.

In addition to being amorphous, some evaporated paroxetine salts produced by the methods described herein, such as paroxetine hydrochloride, may also be characterized as being substantially pure, free-flowing, and/or substantially free of water or another solvent. Where the paroxetine salt preparation includes water, the source of water besides atmospheric moisture in the product is often from the water produced during the esterification process because the starting materials are often substantially free of water. Thus, non-crystalline product may contain up to one equivalent of water. Since water forms an azeotrope with the alcohol used (e.g., ethanol), it is normally removed during distillation and/or evaporation steps with the alcohol. Amorphous salts typically contain less than 10% by weight of an alkanol (e.g., ethanol), and often about 0.01% to about 3% by weight of water or less than 1.5% by weight of water. Non-crystalline salts may be hydrous or anhydrous, and where the non-crystalline salt is amorphous, it is not an anhydrate.

Oven drying temperatures are normally lower than the temperature at which the paroxetine salt in the oven coalesces. The temperature at which a paroxetine salt coalesces can be determined by characterizing the appearance of a paroxetine salt in a standard melting point procedure well known to a person of skill in the art. The pressure for oven drying is between about 40 Torr to about ambient pressure, and often between about 50 Torr and about 100 Torr. The time period for oven drying may be set according to the solvent content of the product. For example, solvent content of an amorphous paroxetine salt composition (e.g., paroxetine hydrogen chloride) is typically about 1.5% or less after drying, and therefore, the amount of time required for oven drying can vary depending upon the amount of solvent in the paroxetine salt composition before it is placed in the oven. Solvent content can be determined using methods well known in the art, and the appropriate time for drying a paroxetine salt composition may be determined, for example, by measuring the solvent content of samples taken from the paroxetine salt composition in the oven during the drying process, and stopping the drying process once the composition reaches a target solvent content.

Formulations of Paroxetine Salts

Paroxetine salts disclosed herein may be optionally formulated with one or more pharmaceutically acceptable carriers and one or more pharmaceutically acceptable excipients, or combinations thereof.

As used herein, the term “pharmaceutically acceptable carrier” can refer to a polymer, including hydroxypropyl cellulose, methyl cellulose, carboxymethyl cellulose, sodium carboxymethyl cellulose, cellulose acetate phthalate, cellulose acetate butyrate, hydroxyethyl cellulose, ethyl cellulose, polyvinyl alcohol, polypropylene, dextrans, dextrins, hydroxypropyl-beta-cyclodextrin, chitosan, co(lactic/glycolid) copolymers, poly(orthoester), poly(anhydrate), polyvinyl chloride, polyvinyl acetate, ethylene vinyl acetate, lectins, carbopols, silicon elastomers, polyacrylic polymers, maltodextrins, lactose, fructose, inositol, trehalose, maltose, raffinose, polyvinylpyrrolidone (PVP), polyethylene glycol (PEG), and alpha-, beta-, and gamma-cyclodextrins, or suitable mixtures of thereof. Examples of polymeric carriers are polyvinylpyrrolidone, hydroxypropylmethyl cellulose, hydroxypropyl cellulose, methyl cellulose, block co-polymers of ethylene oxide and propylene oxide, and polyethylene glycol. Specific examples of polymeric carriers are polyethylene glycol (PEG) having an average molecular weight of from about 1,000 to about 20,000; polyvinylpyrrolidone (PVP) having an average molecular weight of from about 2,500 to about 3,000,000; and PVP having an average molecular weight of from about 10,000 to about 450,000.

Examples of pharmaceutically acceptable excipients are described in Remington's Pharmaceutical Sciences, Mack Publishing Company. Pharmaceutically acceptable excipients include diluents, binders, disintegrants, coloring agents, flavoring agents, lubricants and/or preservatives. Examples of excipients with low moisture content include materials such as dibasic calcium phosphate anhydrous, anhydrous lactose, monosaccharide sugars (e.g., mannitol), disaccharide sugars (e.g., lactitol), powdered cellulose, pregelatinised starch and similar materials. Examples of liquid and semisolid hydrophobic excipients include materials such as polyglycolised glycerides, complex fatty materials of plant origin (e.g., theobroma oil), carnauba wax; plant oils (e.g., peanut, olive, palm kernels, cotton, corn, soya), hydrogenated plant oils (e.g., peanut, palm kernels, cotton, soya, castor, coconut), natural fatty materials of animal origin (e.g., beeswax, lanolin), fatty alcohols (e.g., cetyl, stearyl, lauric, myristic, palmitic, stearic), esters (e.g., glycerol stearate, glycol stearate, ethyl oleate, isopropyl myristate), solid interesterified semi-synthetic glycerides, liquid interesterified semi-synthetic glycerides, amide or fatty acid alcolamides (e.g., stearamide ethanol, diethanolamide of fatty coconut acids), and polyoxyethylene glycols (e.g., PEG 600, PEG 4000).

Paroxetine salts may be administered orally in solid dosage forms, such as tablets, pellets, capsules (e.g., hard or soft gelatin), spheroids, granules, lozenges, powders, and gels. A pharmaceutical composition may be formulated by conventional methods of admixture such as blending, filling, granulation and compressing. These agents may be utilized in a conventional manner, for example in a manner similar to that already used clinically for anti-depressant agents. Where the solid form is granules or pellets, a plurality of granules or pellets may be collected in an aggregation that as a whole constitutes a unit dose. The granules or pellets may be used as a fill for capsules or pressed, optionally with binders or excipients, into tablet form. Gelatin capsules may contain a paroxetine salt and powdered carriers, such as lactose, starch, cellulose derivatives, magnesium stearate, stearic acid, and the like. Similar diluents can be used to manufacture compressed tablets. Both tablets and capsules can be manufactured as sustained release products to provide for continuous release of medication over a period of hours.

Appropriate coatings may be applied to a solid dosage form to increase palatability or delay absorption. Compressed tablets can be sugar coated or film coated to mask any unpleasant taste and protect the tablet from the atmosphere. A tablet may be enteric coated for selective disintegration in the gastrointestinal tract.

Hard gelatin capsules may be prepared by filling standard two-piece hard gelatin capsules each with 10 milligrams of a paroxetine salt, 150 milligrams of lactose, 50 milligrams of cellulose, and 6 milligrams magnesium stearate. Soft Gelatin Capsules may be prepared by mixing a paroxetine salt in a digestible oil such as soybean oil, cottonseed oil or olive oil and injecting the mixture by means of a positive displacement pump into gelatin to form soft gelatin capsules containing 10 milligrams of the paroxetine salt. The capsules may be washed and dried.

Tablets may be prepared by conventional procedures so that the dosage unit was 10 milligrams of a paroxetine salt, 0.2 milligrams of colloidal silicon dioxide, 5 milligrams of magnesium stearate, 275 milligrams of microcrystalline cellulose, 11 milligrams of starch and 98.8 milligrams of lactose. A tablet may also be prepared with the following inactive ingredients: dibasic calcium phosphate dihydrate, hydroxypropyl methylcellulose, magnesium stearate, polyethylene glycols, polysorbate 80, sodium starch glycolate, titanium dioxide, and one or more of following D&C Red No. 30, D&C Yellow No. 10, FD&C Blue No. 2, and FD&C Yellow No. 6. U.S. Pat. No. 5,994,475 discloses other examples of pharmaceutical dosage forms for administration of a paroxetine salt.

A liquid containing a paroxetine salt may be orally administered to a subject. An example of a liquid preparation of a paroxetine salt is an orange-colored and orange flavored liquid containing polacrilin potassium microcrystalline cellulose, propylene glycol, glycerin, sorbitol, methyl paraben, propyl paraben, sodium citrate dihydrate, citric acid anhydrate, sodium saccharin, flavorings, FD&C yellow No. 6 and simethicone emulsion, USP as inactive ingredients. Other liquids containing a paroxetine salt may be prepared and administered to a subject.

Dosages of Paroxetine Salt Formulations

Paroxetine salts may be administered by any means that produce contact of the salt with its site of action, i.e., serotonin re-uptake inhibition, in the body of a mammal. The salts may be administered by any conventional means available for use in conjunction with pharmaceuticals, as individual therapeutic agents or in a combination of therapeutic agents.

The dosage of the novel compounds of this invention administered will vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to about 10 milligrams per kilogram of body weight. The composition may be presented as a unit dose composition containing from 1 to 200 mg, more usually from 5 to 100 mg, for example 10 to 50 mg such as 12.5, 20, 25, or 30 mg. Such composition is normally taken from 1 to 6 times daily, for example 2,3, or 4 times daily so that the total amount of active agent administered is within the range of 5 to 400 mg.

Dosage forms may contain from about 0.1 milligram to about 100 milligrams of active ingredient per unit. In these pharmaceutical compositions the active ingredient will ordinarily be present in an amount of about 0.5-50% by weight based on the total weight of the composition.

Therapeutic Uses for Paroxetine Salt Formulations

Paroxetine salt formulations described herein can be utilized to treat a number of medical conditions. Some medical conditions that may be treated by paroxetine salts include central nervous system conditions, sexual disorders (e.g., premature ejaculation), stress, anxiety, depression, obsessive compulsive disorders, panic disorders, chronic pain, obesity, senile dementia, migraine, bulimia, anorexia, social phobia, pre-menstrual syndrome (PMS), adolescent depression, trichotillomania, dysthymia, and substance abuse (e.g., alcoholism).

The following examples are intended to illustrate but not to limit the invention.

### EXAMPLE 1

### Synthesis of (−)-trans-4R-(4′-fluorophenyl)-3S-[(3′,4′-methylenedioxyphenl)oxymethyl]piperidine (paroxetine free base)

In a 500 ml three-necked round bottom flask equipped with a magnetic stirrer and a dropping funnel was charged N-methyl paroxetine (50.00 g, 145.3 mmol) and toluene (300 ml). The mixture was agitated until solids completely dissolved. Approximately 50 ml toluene was distilled under reduced pressure to dry the remaining toluene solution. A solution of 1-chloroethyl chloroformate (ACECF) (21.80 g, 152 mmol) in 25 ml azeotropically dried toluene was added drop-wise under a nitrogen atmosphere and the temperature of the solution was maintained at about 10° C. in an ice-water bath. The reaction mixture was then allowed to reach room temperature and stirred overnight. After 16 hours, the mixture was filtered and washed with toluene (25 ml). Methanol (45 ml) was added to the mixture and the mixture was stirred for two days (50 hours) at ambient temperature.

150 ml of 2N aqueous potassium hydroxide was added to the mixture and mixed well. The lower aqueous layer was discarded. The upper toluene layer was washed with 50 ml water and 400 ml of water and 8.7 g (145 mmol) of glacial acetic acid were added to it. It was mixed well and layers were allowed to separate. The lower aqueous layer was separated and the upper organic layer was extracted with 100 ml of water. 400 ml of toluene and 150 ml of 2N aqueous potassium hydroxide solution were added to the combined aqueous extract. It was mixed well and layers were allowed to separate. Scum formed in the upper layer. The lower aqueous layer was carefully removed and discarded. The organic layer was filtered whereupon the scum broke and the aqueous layer separated. This aqueous layer was separated and discarded.

The organic extract was dried using 50 g anhydrous sodium sulfate and filtered to yield 450 ml of a clear pale filtrate. The content of pure paroxetine free base in this solution was estimated to be 43.70 g as follows: 50 ml of this solution was evaporated under reduced pressure at a bath temperature of 30° C. to 55° C. to afford a 4.6 g pale thick oil. Thus, a 450 ml solution contained 43.70 g (132 mmols) of free paroxetine base. The separated paroxetine free base was redissolved in the original toluene solution and approximately 150 ml of toluene was distilled under reduced pressure. The final volume of toluene solution was 300 ml and the solution was cooled to 0° C. An HPLC assay determined that the parqxetine base had a purity of 100%.

In another preparation, paroxetine base was purified from 150 g (0.436 mol) (−)-trans-4-(4′-fluorophenyl)-3-methoxy-(3″,4″-methylenedioxyphenyl)-N-methyl piperidine (N-Methyl paroxetine) using other chemicals and reagents proportionately as described above and by removing solvent using a rotory evaporator at reduced pressure and a 45° C. to 60° C. water bath temperature. 126.4 g (88% yield) of pure paroxetine free base was obtained as a pale oil having a purity of 100% according to an HPLC assay.

The free base had the following NMR spectral characteristics:

¹H-NMR (CDCl₃): δ=1.64-1.85 (m, 3H), 2.00-2.12 (m, 1H), 2.58 (dt, 1H), 2.67 (t, 1H), 2.74 (dt, 1H), 3.18 (bd, 1H), 3.39-3.47 (in, 2H), 3.57 (dd, 1H), 5.88 (s, 2H), 6.13 (dd, 1H), 6.34 (d, 1H), 6.62 (d, 1H), 7.94-7.01 (m, 2H), 7.13-7.20 (m, 2H) ppm.

¹³C-NMR (CDCl₃): δ=35.4 (CH₂), 43.0 (CH), 44.6 (CH), 47.1 (CH₂), 50.4 (CH₂), 69.5 (CH₂), 97.9 (CH), 101.0 (CH₂), 105.5 (CH), 107.8 (CH), 115.3 (d, J=21.2 Hz, CH), 128.7 (d, J=7.7 Hz, CH), 140.0 (d, J=3.1 Hz, C), 141.5 (C), 148.1 (C), 154.3 (C), 161.4 (d, J=244 Hz, C) ppm.

Calculated elemental analysis data for Cl₉H₂₀FNO₃ were C 69.29, H 6.12, N 4.25 and the following data were experimentally determined: C 69.50, H 6.13, N 4.12.

### EXAMPLE 2

### Synthesis of (−)-trans-R-(4′-fluorophenl)-3S-[(3′,4′-methylenedioxyphenyl)oxymethyl]piperidine carbonate (paroxetine carbonate)

5.00 g (14.5 mmol) paroxetine free base from Example 1 was dissolved in 30 ml toluene. 75 ml of cold commercial carbonated water (Seltzer water) was added to it. The mixture was mixed well and the layers were allowed to separate. The upper toluene layer was dried using anhydrous sodium sulfate and filtered and the filtrate was diluted with 75 ml of heptane. A thick oil separated from the solution. The solvent was decanted to leave behind 4.0 g of paroxetine carbonate. The oil had a freezing point of 0° C. and a purity of 99.8% according to an HPLC assay.

The oil had the following NMR spectral characteristics:

¹H-NMR (CDCl₃): δ=1.72-1.88 (m, 2H), 2.08-2.20 (m, 1H), 2.63 (dt, 1H), 2.68-2.85 (m, 2H), 3.24 (d, 1H), 3.45 (t, 2H), 3.58 (d, 1H), 3.78 (br, 2H), 5.87 (s, 2H), 6.13 (dd, 1H), 6.34 (d, 1H), 6.62 (d, 1H), 6.93-7.01 (m, 2H), 7.13-7.21 (m, 2H) ppm. Signals at 0.9, 1.3, 2.36 and 7.1-7.3 were attributed to residual toluene and heptane in the sample.

¹³C-NMR (CDCl₃): δ=34.6 (CH₂), 42.4 (CH), 44.2 (CH), 46.5 (CH₂), 49.7 (CH₂), 69.3 (CH₂), 97.9 (CH), 101.0 (CH₂), 105.5 (CH), 107.8 (CH), 115.4 (d, J=21.0 Hz, CH), 128.7 (d, J=7.7 Hz, CH), 139.6 (d, J=2.9 Hz, C), 141.5 (C), 148.1 (C), 154.3 (C), 161.5 (d, J=244 Hz, C) ppm. Signals at 14.1, 21.5, 22.6, 29.0, 31.8, 125.2, 128.1, 129.0, and 138.0 were attributed to the residual toluene and heptane in the sample.

Calculated elemental analysis data for C₃₉H₄₂F₂N₂O₉ were C 65.00, H 5.87, N 3.89 and the following data was experimentally determined: C 66.65, H 6.28, N 3.49. The slightly higher carbon and hydrogen content was correlated to the presence of toluene and heptane in the sample.

### EXAMPLE 3

### Synthesis of (−)-trans-4R-(4′-fluorophenyl)-3S-[(3′,4′-methylenedioxyphenyl)oxymethyl]piperidine phosphate (paroxetine dihydrogen phosphate)

5.00 g (14.5 mmole) paroxetine free base from Example 1 was dissolved in 35 ml of absolute ethanol. A solution of 1.66 g (14.5 mmol) 85% phosphoric acid in 5 ml ethanol was added to the mixture at room temperature and mixed well. A thick gum separated. The mixture was warmed to about 60° C. to 70° C. with mixing and an off-white solid separated. The mixture stood at room temperature overnight and then filtered and washed with 10 ml of ethanol. Drying in air overnight at 50° C. yielded 5.4 g (84% yield) paroxetine phosphate as an off-white solid, having a melting point of 203° C. to 208° C., and a purity of 99.5% according to an HPLC assay.

The solid had the following NMR spectral characteristics:

¹H-NMR (DMSO-d6): δ=1.74 (d, 1H), 2.12 (q, 1H), 2.48 (m, 1H), 2.74-2.93 (m, 3H), 3.35 (d, 1H), 3.38-3.56 (m, 2H), 5.89 (s, 2H), 6.08 (dd, 1H), 6.37 (d, 1H), 6.67 (d, 1H), 7.10 (m, 2H), 7.30 (m, 2H) ppm.

¹³C-NMR (DMSO-d₆): δ=30.2 (CH₂), 38.6 (CH), 41.3 (CH), 43.4 (CH₂), 45.8 (CH₂), 68.2 (—CH₂), 97.8 (—CH—), 100.9 (—CH₂), 105.5 (—CH), 107.9 (—CH), 115.2 (d, J=20.9 Hz, —CH), 129.2 (d, J=7.7 Hz, —CH), 139.1 (d, J=2.7 Hz, —C—), 141.2 (—C—), 147.8 (—C—), 153.7 (—C—), 160.9 (d, J=242.5 Hz, —C—) ppm.

Calculated elemental analysis data for C₁₉H₂₃FNO₇P were C 53.40, H 5.42, N 3.28 and the following data were experimentally determined: C 53.14, H 5.36, N 3.28

### EXAMPLE 4

### Synthesis of (−)-trans-4R-(4′-fluorophenyl)-3S-[(3′,4′-methylenedioxyphenyl)oxymethyl]piperidine sulfate (paroxetine sulfate)

From 5.00 g (14.5 mmole) paroxetine free base of Example 1 and 0.75 g (7.27 mmoles) of 95% concentrated sulfuric acid and following the procedure according to Example 3, there was obtained 4.1 g (71% yield) paroxetine sulfate as an off-white solid having a melting point of 240° C. to 245° C. and having a purity of 100% according to an HPLC assay.

The solid had the following NMR spectral characteristics:

¹H-NMR (DMSO-d₆): δ=1.75 (d, 1H), 2.13 (q, 1H), 2.48 (m, 1H), 2.71-2.91 (m, 3H), 3.30 (d, 1H), 3.40-3.53 (m, 2H), 5.89 (s, 2H), 6.08 (dd, 1H), 6.37 (d, 1H), 6.67 (d, 1H), 7.10 (m, 2H), 7.30 (m, 2H) ppm.

¹³C-NMR (DMSO-d₆): δ=30.9 (CH₂), 39.3 (CH), 41.7 (CH), 44.1 (CH₂), 46.7 (CH₂), 68.4 (—CH₂), 97.7 (—CH—), 101.0 (—CH₂), 105.5 (—CH), 107.9 (—CH), 115.2 (d, J=20.8 Hz, —CH), 129.2 (d, J=7.9 Hz, —CH), 139.4 (d, J=2.8 Hz, —C—), 141.2 (—C—), 147.9 (—C—), 153.7 (—C—), 160.9 (d, J=242.3 Hz, —C—) ppm.

Calculated elemental analysis data for C₃₈H₄₂F₂N₂O₁₀S were C 60.31, H 5.59, N 3.70 and the following data were experimentally determined: C 60.22, H 5.54, N 3.70.

### EXAMPLE 5

### Synthesis of (−)-trans-R-(4′-fluorophenyl)-3S-[(3′,4′-methylenedioxyphenyl)oxymethyl]piperidine trifluoroacetate (paroxetine trifluoroacetate)

6.20 g (18.0 mmoles) paroxetine free base from Example 1 was dissolved in 45 ml toluene. A solution of 2.03 g (17.6 mmoles) 99% trifluoroacetic acid in 2 ml of toluene was added to it at room temperature, mixed well and placed in a deep freezer at −10° C. overnight. A solid separated and was filtered, washed with 10 ml of a 6:4 mixture of toluene/heptane, and dried in air overnight to yield 4.0 g (48% yield) paroxetine trifluoroacetate as a white solid, having a melting point of 116° C. to 117° C. and having a purity of 100% according to an HPLC assay. Cooling the filtrate overnight yielded a product having a melting point of 114 to 116° C.

The compound had the following NMR spectral characteristics:

¹H-NMR (DMSO-d₆): δ=1.88 (d, 1H), 1.96 (dq, 1H), 2.39 (m, 1H), 2.88 (dt, 1H), 3.00 (q, 2H), 3.40(d, 1H), 3.47-3.62 (m, 3H), 5.92 (s, 2H), 6.19 (dd, 1H), 6.48 (d, 1H), 6.73 (d, 1H), 7.15 (m, 2H), 7.24 (m, 2H) ppm.

¹³C-NMR (DMSO-d₆): δ=30.0 (CH₂), 38.5 (CH), 40.8 (CH), 43.5 (CH₂), 45.8 (CH₂), 67.9 (—CH₂), 97.8 (—CH—), 101.0 (—CH₂), 105.7 (—CH), 107.9 (—CH), 115.4 (d, J=21.2 Hz, —CH), 117.1 (q, J=299 Hz, CF₃), 129.0 (d, J=7.9 Hz, —CH), 138.6 (d, J=2.9 Hz, —C—), 141.4 (—C—), 147.8 (—C—), 153.6 (—C—), 158.6 (q, J=31.5 Hz, CF₃COO), 161.0 (d, J=242.5 Hz, —C—) ppm.

Calculated elemental analysis data for C₂₁H₂₁F₄NO₅ were C 56.89, H 4.77, N 3.16 and the following data were experimentally derived: C 56.61, H 4.53, N 3.08.

### EXAMPLE 6

### Synthesis of (−)-trans-4R-(4′-fluorophenyl -3S-[(3′,4′-methylenedioxyphenyl)oxymethyl]piperidine formate (paroxetine formate)

To 300 ml toluene solution containing 43.70 g (132 mmols) of free paroxetine base prepared according to Example 1, was added 5.83 g (126 mmols) of formic acid (96% purity) in 30 ml of toluene and mixed thoroughly. The solution was allowed to stand at 0° C. for one hour and agitated gently for a few seconds. It was allowed to stand at the same temperature for an additional two hours. The whole solution filled with crystalline solids. The solids were filtered and washed two times with 50 ml of 6:4 mixture of toluene and heptane. The solid was dried overnight at ambient temperature which yielded 44.0 g of a white solid which was further dried at 50° C. overnight to afford 43.0 g (79%) paroxetine formate as a crystalline solid, with a melting point of 100-101° C., a purity of 100% by a HPLC assay, and an optional rotation of [α]²⁵_{D}=−86.1 in 1% ethanol. Related substances and impurities were found not detected by HPLC analysis.

The product was characterized by NMR. The middle peak from solvent signal was used as a chemical shift reference. Assignments are based on a DEPT experiment.

¹H-NMR (DMSO-d₆): δ=1.74-1.81 (m, 1H), 1.83-1.96 (m, 1H), 2.25-2.36 (m, 1H), 2.72-2.87 (m, 3H), 3.20-3.27 (d, 1H), 3.36-3.43 (dd, 1H), 3.44-3.50 (dd, 1H), 3.52-3.57 (dd, 1H), 5.92 (s, 2H), 6.17 (dd, 1H), 6.46 (d, 1H), 6.72 (d, 1H), 7.10-7.17 (m, 2H), 7.22-7.28 (m, 2H), 8.43 (s, 1H) ppm.

¹³C-NMR (DMSO-d₆): δ=31.5 (CH₂), 41.9 (CH), 44.0(CH₂), 46.7 (CH₂), 68.4 (—CH₂), 97.8 (—CH—), 101.0 (—CH₂), 105.5 (—CH), 107.9 (—CH), 115.3 (d, J=21 Hz, —CH), 129.0 (d, J=8.0 Hz, —CH), 139.4 (—C—), 141.2 (—C—), 147.8 (—C—), 153.8 (—C—), 160.9 (d, J=242 Hz, —C—), 165.7 (HCOOH) ppm.

¹³C-NMR (CDCl₃): δ=30.9 (CH₂), 38.9 (CH), 42.4 (CH), 44.3 (CH₂), 46.9 (CH₂), 68.2 (CH₂), 98.1 (CH), 101.2 (CH₂), 105.9 (CH), 108.0 (CH), 115.8 (d, J=21.2 Hz, CH), 129.0 (d, J =7.9 Hz, CH), 137.8 (d, J=3.4 Hz, C), 141.2 (C), 148.4 (C), 154.0 (C), 162.0 (d, J=242 Hz, C), 169.0 (HCOOH) ppm.

Elemental analysis results of C 63.99, H 5.91, F 5.06, and N 3.73 were calculated for C₂₀H₂₂FNO₅, and the following elemental analysis results were identified experimentally: C 64.16, H 5.90, F 4.93, N 3.73.

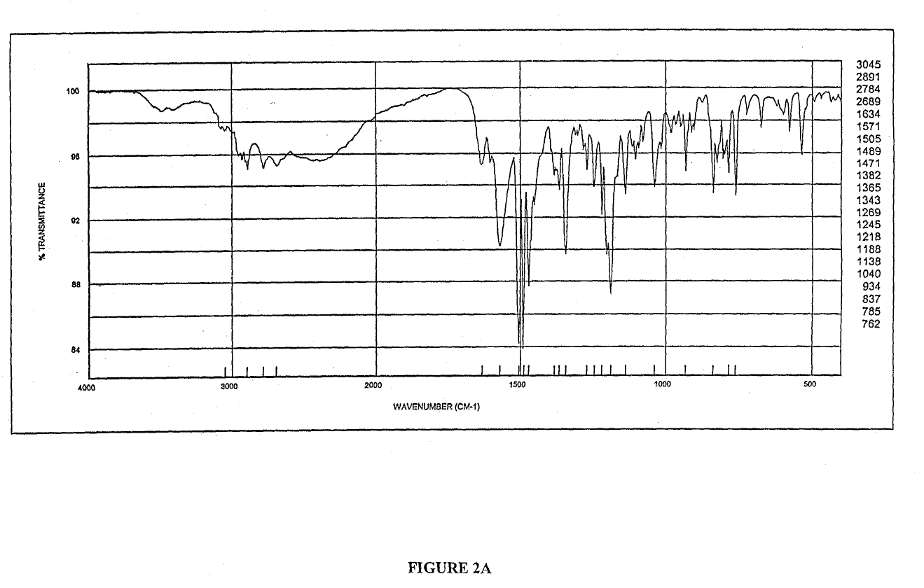

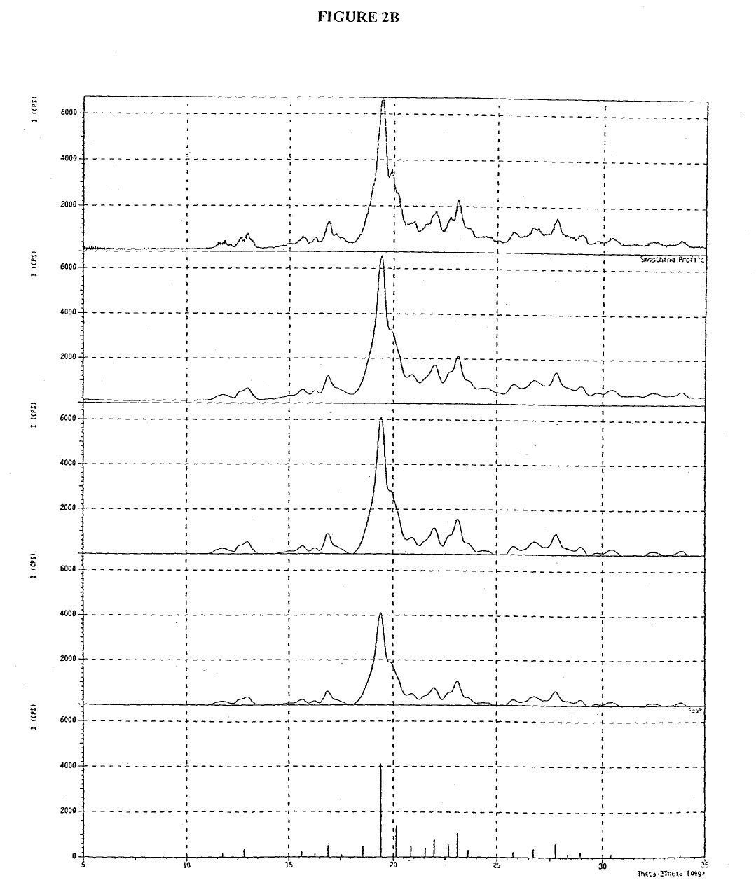

An FT-IR (Attenuated Total Reflectance) analysis of the product was performed using a Nicolet Nexus 470 FT-IR specrometer. The following bands (cm⁻¹) were observed: 762, 785, 837,934, 1040, 1138, 1188, 1218, 1245, 1269,1343, 1365, 1382, 1471, 1489, 1505, 1571, 1634, 2689, 2784, 2891, and 3045 (FIG. 2A).

An X-ray powder diffraction study was carried out by loading a powder specimen into a Schimadzo XRD-6000 diffractometer. The measurement conditions were as follows: X-ray tube target Cu (voltage 40.0 kV and current 40 mA); slits (divergence slit: 1.00000 (deg), scatter slit: 1.00000 (deg), receiving slit: 0.15000 (mm)); scanning (drive axis: Theta-2Theta, scan range 5.0000-35.0000 (deg); scan mode: continuous scan, scan speed 2.0000 (deg/min); sample pitch: 0.0200 (deg), present time: (0.60 sec). An X-ray diffractogram is shown in FIG. 2B and the major peaks for that diffractogram are listed in Table 1.

TABLE 1
Major peaks in paroxetine formate

| 2 Theta | Intensity | |
|---|---|---|
| (deg.) | (counts) | I/I1 |
| 19.3951 | 2473 | 100 |
| 20.1400 | 841 | 34 |
| 23.0800 | 635 | 26 |

### EXAMPLE 7

### Synthesis of (−)-trans-4R-(4′-fluorophenyl)-3S-[(3′,4′-methylenedioxyphenyl)oxymethyl]piperidine acetate (paroxetine acetate)

In a 250 ml round bottom flask equipped with a magnetic stirrer and a dropping funnel was charged N-methyl paroxetine (5.00 g, 14.53 mmol), bis-1,8-(dimethyl amino) naphthalene (“Proton Sponge,” 0.623 g, 2.91 mmol), and toluene (25 ml). The mixture was agitated until solids completely dissolved. A solution of 1-chloroethyl chloroformate (ACECF) (2.30 g, 16.08 mmol) in 5 ml toluene was added drop-wise in a nitrogen atmosphere while maintaining the temperature of the solution below 20° C. in an ice-water bath. Some precipitation occurred. The reaction mixture was then allowed to reach room temperature and stirred overnight. After 24 hours, TLC analysis showed the reaction was complete. The color of the mixture was light orange.

Hydrogen chloride gas was bubbled through the reaction mixture for 30 second at which time a white precipitate occurred. Only about 0.1 g hydrogen chloride is needed for this conversion. Too much hydrogen chloride at this stage will cause decomposition of material during the next methanolysis stage, which requires two days of stirring at ambient temperature.

The mixture was filtered through celite and rinsed with toluene (5 ml). The filtrate was a bit turbid. It was transferred to a 250 ml round bottom flask, and washed with 5 ml toluene. Approximately 10 to 20% of the solvent was distilled under reduced pressure in a water bath at a temperature of below 40° C. This procedure substantially removed any additional hydrogen chloride gas from the mixture, thus making the intermediate less susceptible to decomposition. The volume of the final solution was 25 ml. Methanol (4.5 ml) was added to the mixture and the mixture was stirred over a weekend (63 hours) at ambient temperature. TLC analysis showed the reaction was complete.

25 ml 2N aqueous potassium hydroxide was added to the mixture and it was mixed well. The layers separated quickly. The lower aqueous layer was discarded.

40 ml water and 0.87 g glacial acetic acid were added to the resulting mixture. It was mixed well and layers were allowed to separate. The layers separated quickly and the upper organic layer was discarded. 35 ml toluene and 25 ml 2N aqueous potassium hydroxide solution were added to the lower aqueous layer. It was mixed well and the layers were allowed to separate. Some foaming at the bottom and sides of the upper layer remained, and never dissipated. The lower aqueous layer was carefully removed and discarded.

The upper organic layer was then dried using MgSO₄. The resulting mixture was evaporated to dryness under reduced pressure in a water bath having a temperature of about 40° C. to afford 4.16 g (12.61 moles, 86% yield) paroxetine free base as pale yellow oil. The paroxetine free base was determined to have a purity of 100% by an HPLC assay (150×4.6(ID) mm ODS-AM Column, particle size 125 μm at 22° C.; 10 mM NaClO₄ mobile phase and acetonitrile binary gradient from 25% to 65%, detection at 250±10 nm).

This oil was dissolved in 30 ml toluene and a solution of 0.72 g (12.0 mmol, 0.95 equivalents) glacial acetic acid in 5 ml toluene was added to it. The solution was mixed, seeded and allowed to stand at 5° C. in a refrigerator. After two hours, the solid was separated and broken and the mixture was disturbed in order to facilitate more crystallization. The flask was placed again in the refrigerator overnight. The mixture was not stirred or agitated to facilitate seeding. Solid was filtered and washed two times with a 5 ml toluene/hexane (6:4) mixture. It was dried in air to yield 4.0 g paroxetine acetate as a white crystalline solid, with an overall yield of 70% and an 81% yield from purified base. The product had a melting point of 124° C. to 125° C., and an optical rotation of [α]²⁵_{D}=81.2°. An HPLC assay indicated a purity of 100.2%.

### EXAMPLE 8

### Synthesis of (−)-trans-4R-(4′-fluorophenyl)-3S-[(3′,4′-methylenedioxyphenyl)oxymethyl]piperidine acetate (paroxetine acetate)

In a 500 ml three necked round bottom flask equipped with a magnetic stirrer and a dropping funnel was charged N-methyl paroxetine (50.00 g, 145.3 mmol), bis-1,8-(dimethyl amino) naphthalene (“Proton Sponge”) (6.25 g, 29.1 mmol), and toluene (250 ml). The mixture was agitated until solids completely dissolved. A solution of 1-chloroethyl chloroformate (ACECF) (22.30 g, 156 mmol) in 25 ml toluene was added drop-wise in a nitrogen atmosphere with the temperature of the solution maintained at about 10° C. in an ice-water bath. The reaction mixture was then allowed to reach room temperature and stirred overnight. After 20 hours, TLC analysis showed the reaction was complete.

A solution of hydrogen chloride gas in diethyl ether (19.2 ml, 1.52M, 29 mmol hydrogen chloride) was added to the reaction mixture and stirred for 10 minutes. The mixture was filtered and washed with toluene (25 ml). The clear filtrate was transferred to a 5L round bottom flask and approximately 10 to 20% of the solvent was distilled under reduced pressure in a water bath having a temperature of below 40° C. The volume of the final solution was measured to be 260 ml. Methanol (45 ml) was added to the mixture and the mixture was stirred over a weekend (63 hours) at ambient temperature. TLC analysis showed the reaction was complete.

150 ml 2N aqueous potassium hydroxide was added and mixed well. The lower aqueous layer was discarded. The upper toluene layer was washed with 50 ml of water. The toluene layer contained crude paroxetine free base.

400 ml water and 8.7 g (145 mmol) glacial acetic acid were added to the mixture. It was mixed well and layers were allowed to separate. The upper organic layer was extracted with 100 ml of water and the aqueous extracts containing paroxetine acetate were combined. The organic layer was discarded. To the combined aqueous layer which contained paroxetine acetate was added 300 ml toluene and 150 ml 2N aqueous potassium hydroxide solution. The mixture was mixed well and layers were allowed to separate. Scum formed at the junction of the two layers. The lower aqueous layer was carefully removed and extracted with 100 ml toluene. The extract was combined with the upper organic layer.

The combined organic extract was dried using anhydrous MgSO₄ (50 g) and filtered to yield 450 ml of clear pale filtrate. The content of pure paroxetine free base in this solution was estimated to be 43.13 g as follows: 50 ml of this solution was evaporated under reduced pressure at a 55° C. bath temperature to afford 4.57 g of a pale thick oil. Thus a 450 g solution contained 43.13 g or 124.6 mmols of the base. The separated paroxetine free base was redissolved in the original toluene solution and approximately 150 ml toluene was distilled under reduced pressure. The final volume of toluene solution was 300 ml and the solution was cooled to 0° C. in an ice water bath. An HPLC assay determined that the paroxetine base had a purity of 100%.

7.18 g (118.4 mmols) glacial acetic acid in 30 ml toluene was added and mixed thoroughly. The solution was allowed to stand at the same temperature for one hour and agitated gently for a few seconds and then was allowed to stand at the same temperature in an ice water bath for an additional two hours. The solution filled with crystalline solids. The solids were filtered and washed two times with 50 ml of a 6:4 mixture of toluene and heptane. Drying in air overnight at an ambient temperature yielded 44.4 g of white solid which was further dried at 60° C. overnight to afford 44.4 g paroxetine acetate as a white crystalline solid. The white solid had a melting point of 124° C. to 125° C., a purity of 101.4% according to HPLC analysis, and an optical rotation of [α]²⁵_{D}=−81.5°. The process provides a 78% overall yield from N-methyl paroxetine and a 87% yield on the basis of purified free base.

Elemental analysis results of C 64.77, H 6.21, F 4.88, N 3.60 were calculated for C₂₁H₂₄FNO₅, and the following elemental analysis results were identified experimentally: C 64.72, H 6.19, F 4.93, N 3.51.

The product was characterized by NMR. The middle peak from the solvent signal was used as a chemical shift reference. Assignments are based on a DEPT experiment.

¹H-NMR (DMSO-d₆): δ=1.63-1.76 (m, 2H), 1.85 (s, 3H), 2.02-2.14 (m, 1H), 2.55 (t, 1H), 2.57-2.68 (m, 2H), 3.06 (d, 1H), 3.27 (dd, 1H), 3.41-3.52 (m, 2H), 5.92 (s, 2H), 6.15 (dd, 1H), 6.44 (d, 1H), 6.72 (d, 1H), 7.08-7.15 (m, 2H), 7.22-7.29 (m, 2H) ppm.

¹³C-NMR (DMSO-d₆): δ=22.1 (CH₃), 33.9 (CH₂), 41.2 (CH), 43.3 (CH), 45.6 (CH₂), 48.8 (CH₂), 69.1 (—CH₂), 97.7 (—CH—), 100.9 (—CH₂), 105.4 (—CH), 107.9 (—CH), 115.1 (d, J=21.0 Hz, —CH), 129.0 (d, J=7.8 Hz, —CH), 140.3 (d, J=2.8 Hz, —C—), 141.1 (—C—), 147.8 (—C—), 153.9 (—C—), 160.7 (d, J=241.6 Hz, —C—), 172.8 (—COO⁻) ppm.

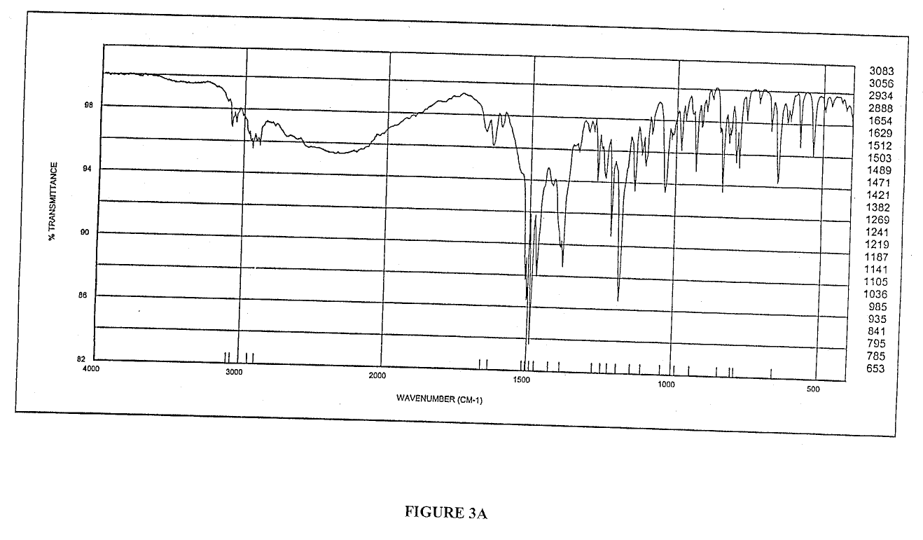

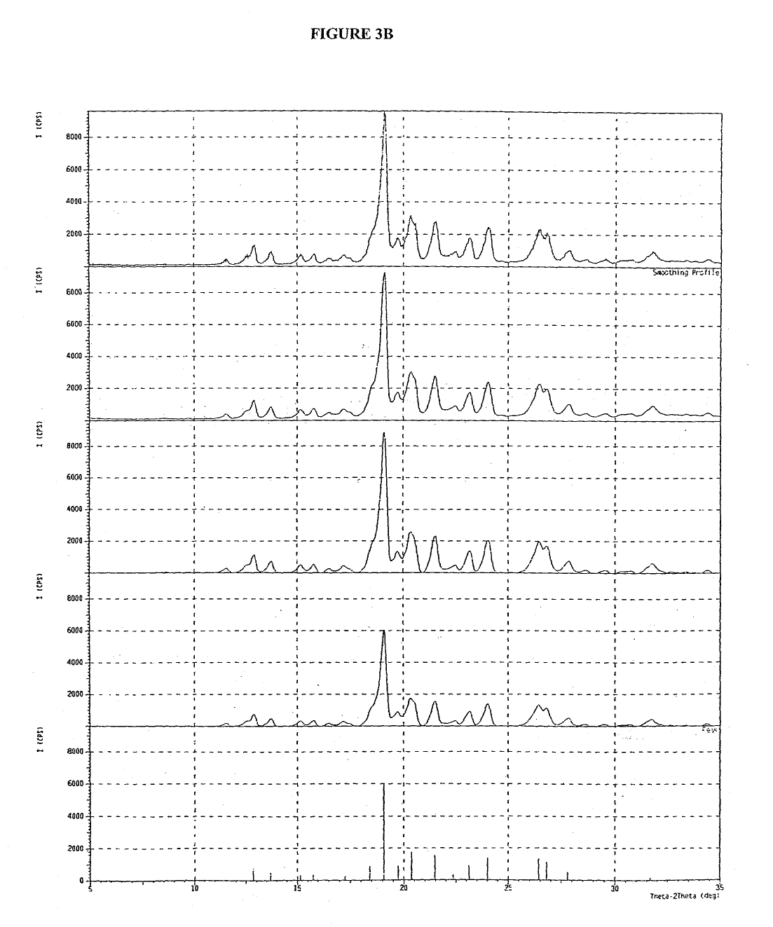

An FT-IR (attenuated total reflectance) analysis of the product was carried out using a Nicolet Nexus 470 FT-IR spectrometer. The following bands (cm⁻¹) were observed: 653, 785, 795, 841, 935, 985, 1036, 1105, 1141, 1187, 1219, 1241, 1269, 1382, 1421, 1471, 1489, 1503, 1512, 1629, 1654, 2888, 2934, 3056, and 3083 (FIG. 3A).

An X-ray powder diffraction study was carried out by loading a powder specimen into a Schimadzo XRD-6000 diffractometer. The measurement conditions were as follows: X-ray tube target Cu (voltage 40.0 kV and current 40 mA); slits (divergence slit: 1.00000 (deg), scatter slit: 1.00000 (deg), receiving slit: 0.15000 (mm)); scanning (drive axis: Theta - 2Theta, scan range 5.0000-35.0000 (deg); scan mode: continuous scan, scan speed 2.0000 (deg/min); sample pitch: 0.0200 (deg), present time: (0.60 sec). An X-ray diffractogram is shown in FIG. 3B and the major peaks for that diffractogram are listed in Table 2.

TABLE 2
Major peaks in paroxetine acetate

| 2 Theta | Intensity | |
|---|---|---|
| (deg.) | (counts) | I/I1 |
| 19.0771 | 3622 | 100 |
| 20.3742 | 1050 | 29 |
| 21.4833 | 938 | 26 |

### EXAMPLE 9

### Synthesis of (−)-trans-4R-(4′-fluorophenyl)-3S-[(3′,4′-methylenedioxyphenyl)oxymethyl]piperidine acetate (paroxetine acetate)

Following the procedure according to Example 8, starting with 1.0 kg N-methyl paroxetine, using 31.3 g proton sponge, 448 g 1-chloroethyl chloroformate, 90 ml (1.52M) hydrogen chloride in ether, 900 ml methanol, and other reagents proportionally, there was isolated 865 g of paroxetine acetate as an off-white crystalline solid, having a melting point of 124-125° C., a purity of 101.40% according to HPLC analysis, and an optical rotation of [α]²⁵_{D}=−81.5° in 1% methanol. The overall yield was 77% and the yield on the basis of purified free base was 90%.

### EXAMPLE 10

### Synthesis of (−)-trans-4R-(4′-fluorophenyl-3S-[(3′,4′-methylenedioxyphenyl)oxymethyl]piperidine acetate (paroxetine acetate)

The following example demonstrates conversion to paroxetine acetate in substantially low yield when purification steps are not carried out prior to acetate salt formation. In a 250 ml three necked round bottom flask equipped with a magnetic stirrer, and a dropping funnel was charged N-methyl paroxetine (5.00 g, 14.53 mmol) and dry toluene (25 ml). The mixture was agitated until solids completely dissolved. A solution of 1-chloroethyl chloroformate (ACECF) (2.30 g, 16.08 mmol) in 5 ml dry toluene was added drop-wise under a nitrogen atmosphere where maintaining the temperature of the solution at about 10° C. in an ice-water bath. The reaction mixture was then allowed to reach room temperature and was stirred overnight. Methanol (4.5 ml) was added to the mixture and the mixture was stirred over a weekend (66 hours) at ambient temperature. 20 ml of 2N aqueous potassium hydroxide was added and mixed well. The lower aqueous layer was discarded. The upper organic layer was dried over anhydrous sodium sulfate, filtered and evaporated under reduced pressure to yield 5.41 g of thick oil. This oil was dissolved in 25 ml of dry toluene and 0.87 g (14.5 mmoles) glacial acetic acid in 5 ml toluene was added. The total volume was made 40 ml by the addition of more toluene, seeded and placed in a refrigerator at 0° C. for two hours. The crystals were separated and scratched and mixed well and the mixture was again allowed to stand at the same temperature overnight. Solids were filtered and washed two times with a 6:4 mixture of toluene and heptane. The solid was dried in air overnight to yield 2.26 g (39%) paroxetine acetate as an off white solid having a melting point of 123° C. to 124° C.

### EXAMPLE 11

### Synthesis of (−)-trans-4R-(4′-fluorophenyl)-3S-[(3′,4′-methylenedioxyphenyl)oxymethyl]piperidine hydrochloride, amorphous (amorphous paroxetine hydrochloride) from paroxetine formate

25 g (66.5 mmoles) paroxetine formate was added slowly, with stirring, to a mixture of ethanol (250 ml) and a 10% solution of hydrogen chloride in ethanol (25.5 g, 2.55 g hydrogen chloride). The resultant pale solution was evaporated under reduced pressure (using house vacuum) in a rotary evaporator at 40° C. to a pale oil. This was immediately subjected to a high vacuum (0.5 to 1 Torr) at a 40° C. bath temperature for two hours to yield homogenous foam. This foam was carefully removed from the flask to yield 23.1 g (95%) amorphous paroxetine hydrochloride as white powder, with a melting point of 65° C. to 85° C. (starts shrinking at 64° C., starts droplet formation at 71° C., 85 becomes a transparent gel at 85° C., and the gel starts moving and settles at 130-140° C.), as purity of 100% by an HPLC assay, and an optical of [α]²⁵_{D}=−86.3 in 1% ethanol. Related substances and impurities were below detection limit by HPLC analysis

The solid was characterized by NMR and the resulting spectra had characteristic absorption signals that were consistent with that of the compound produced in Example 12. The solid displayed the characteristic halo associated with an amorphous solid by x-ray powder diffraction analysis. Signals due to crystalline solid were absent.

### EXAMPLE 12

### Synthesis of (−)-trans-4R-(4′-fluorophenyl)-3S-[(3′,4′-methylenedioxyphenyl)oxymethyl]piperidine hydrochloride, amorphous (amorphous paroxetine hydrochloride) from paroxetine acetate

40 g (102.6 mmoles) paroxetine acetate was added slowly, with stirring, to a mixture of ethanol (400 ml) and a 10.81% solution of hydrogen chloride in ethanol (140 g, 15.134 g hydrogen chloride). The resultant pale solution was evaporated under reduced pressure (using house vacuum) in a Buchi rotary evaporator (model R-121) at 40° C. to a pale oil. House vacuum can range from about 40 Torr to about 90 Torr. This oil was immediately subjected to a high vacuum (0.1 to 1 Torr) at a 40° C. bath temperature for two hours to yield homogenous foam. This foam was carefully removed from the flask and dried overnight at 50° C. to yield 36.0 g amorphous paroxetine hydrochloride as a light pink powder, having a melting point of 68° C. to 88° C. (starts shrinking at 68° C., droplet formation starts at 71° C., becomes a transparent gel at 88° C., appears to be liquefying at 110° C. to 128° C.), a purity of 100.8% by HPLC analysis, and [α]²⁵_{D}=−86.0° in 1% ethanol. The yield was 95%. Related substances and impurities were below detection limits when analyzed by HPLC.

X-ray powder diffraction data was obtained by a Schimadzo XRD-6000 instrument. The data showed a characteristic halo associated with the amorphous compound, as can be seen in FIG. 4. Measurement conditions were as follows: X-ray tube (target: Cu; voltage: 40.0 (kV); current: 30.0 mA); slits (divergence slit: 1.00000 (deg); scatter slit: 1.00000 (deg); receiving slit: 0.15000 (mm)); scanning (drive axis: Theta-2Theta; scan range: 5.0000-44.9800 (deg); scan mode: continuous scan; scan speed: 2.0000 (deg/min); sampling pitch: 0.0200 (deg); preset time: 0.60 (sec)).

The product was also characterized by NMR. The middle peak from the solvent signal was used as a chemical shift reference. Assignments are based on a DEPT experiment.

¹H-NMR (DMSO-d₆): δ=1.80-1.88 (d, 1H), 2.08-2.21 (m, 1H), 2.52-2.63 (bm, 1H), 2.82-3.02 (m, 3H), 3.32-3.40 (d, 1H), 3.45-3.53 (m, 2H), 3.55-3.61 (m, 1H), 5.92 (s, 2H), 6.17 (dd, 1H), 6.47 (d, 1H), 6.71 (d, 1H), 7.10-7.17 (m, 2H), 7.20-7.28 (m, 2H), 9.45 (b, 2H) ppm.

¹³C-NMR (DMSO-d₆): δ=29.8 (CH₂), 38.3 (CH), 40.8(CH), 43.4 (CH₂), 45.7 (CH₂), 68.0 (—CH₂), 97.8 (—CH—), 101.0 (—CH₂), 105.8 (—CH), 107.9 (—CH), 115.5 (d, J=20 Hz, —CH), 129.0 (d, J=7.8 Hz, —CH), 138.8 (d, J=2.9 Hz, —C—), 141.3 (—C—), 147.9 (—C—), 153.7 (—C—), 161.0 (d, J=242 Hz, —C—) ppm.

### EXAMPLE 13

### Synthesis of (−)-trans-4R-(4′-fluorophenyl)-3S-[(3′,4′-methylenedioxyphenyl)oxymethyl]piperidine hydrochloride, amorphous (amorphous paroxetine hydrochloride) from paroxetine acetate

Paroxetine acetate (400 g, 1.03 moles) was added slowly to a stirring mixture of absolute ethanol (3.6 liters) and 10.5% w/w solution of hydrogen chloride in absolute ethanol (392.5 g). The pale solution was stirred for 10 minutes. The solvent was distilled under reduced pressure (71 Torr) in a Buchi rotary evaporator model R-153 at a 43° C. bath temperature at about 100 rpm to a pale oil. After sixty minutes, about 3.5 liters of solvent was distilled. At this point an additional vacuum pump (Welch) was coupled with the existing Buchi vacuum pump. The pressure in the system decreased to about 30 Torr in about ten minutes and the thick oil which formed in the flask began foaming. In the next twenty minutes, a pressure of 15 Torr was achieved by adjusting appropriate valves and isolating distilled solvent in the receivers. The thick oil in the flask expanded between 20 Torr and 15 Torr pressure at a 43° C. bath temperature to a shining off-white foam solid which filled the entire 20 liter flask. It was continued to be rotory evaporated for about one hour further at same temperature and pressure. The foam was blended and carefully removed from the flask to provide 376 g of off-white shining powder, having a melting point of about 100° C. (at 52° C. the solid started to shrink; at 66° C. to 68° C. there was a transition to glass; at 100° C. to 114° C. the glass moved to the bottom; at 118° C. to 160° C. there was bubbling, and at 165° C. the glass liquefied). The powder had an optical rotation of [α]²⁵_{D}=−84.4 at 1% in ethanol.

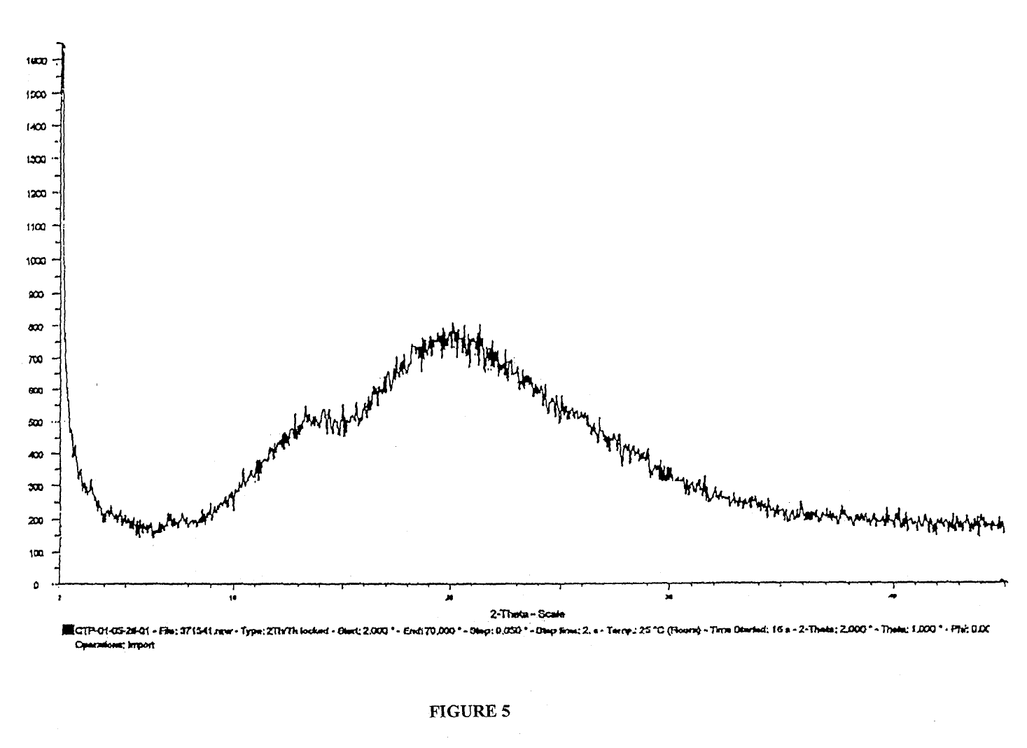

An X-ray powder diffraction study carried out by loading powder specimens into a plastic dish placed in a Siemens D5000 diffractometer showed the characteristic halo associated with amorphous compounds (FIG. 5). The samples were X-rayed from 2 to 70 degrees two theta using copper radiation with the sample spinning in an accelerating voltage of 40 kV/30 mA, a step size of 0.05 degrees, and a data acquisition time of 2.0 seconds per step. A peak search routine was used to determine the positions of the peaks, which were then edited visually.

A sample (10.721 g) of the above amorphous paroxetine hydrochloride was dried in a vacuum oven for 16 hours at 40° C. at reduced pressure to yield 10.419 g (0.302 g reduction in weight) of a free-flowing off-white powder, having an optical rotation of [α]²⁵_{D}=−87.2 at 1% in ethanol and having a melting point of about 100° C. (at 66-68° C. the powder started to shrink; at 76° C. the powder began to melt; at 82° C. the powder began a transition to glass; at 86-88° C. the transition to transparent glass was complete; at 115° C. the glass started moving to the bottom; at 133-135° C. the movement was complete; and at 165-170° C. there was a clear liquid).

### EXAMPLE 14

### Synthesis of (−)-trans-4R-(4′-fluorophenyl)-3S-[(3′,4′-methylenedioxyphenyl)oxymethyl]piperidine hydrochloride, amorphous (amorphous paroxetine hydrochloride) from paroxetine acetate

A similar experiment as described in Example 13 starting with 400 g paroxetine acetate yielded similar results. The foam solid was formed between 20 and 11 Torr pressure at a 44° C. water bath temperature in a Buchi R-153 rotory evaporator.

### EXAMPLE 15

### Synthesis of (−)-trans-4R-(4′-fluorophenyl)-3S-[(3′,4′-methylenedioxyphenyl)oxymethyl]piperidine hydrochloride amorphous (amorphous paroxetine hydrochloride) from paroxetine acetate

An 11% (w/w) solution of hydrogen chloride in ethanol was prepared in a fume hood. In a round bottom (1 L) flask fitted with a pressure release vent and equipped with a stir bar, ethyl alcohol (400.0 g) was charged and the tare weight of the flask containing ethyl alcohol and stir bar was recorded. The flask was equipped with gas inlet/outlet adapter and placed in an ice water bath (200-300 g ice) on a magnetic stirring plate. Using tygon tubes, the hydrogen chloride cylinder was connected to an empty trap from which another tube was directly connected to the gas adapter equipped on the round bottom flask. The gas outlet was connected to a mineral oil trap. While being gently stirred, the hydrogen chloride valve was slowly opened sparging 41.2 grams of anhydrous hydrogen chloride gas through the ethyl alcohol. After about 5 minutes the hydrogen chloride was turned off and the contents of the flask were weighed. This process was continued until the weight increased by about 49 g. The hydrogen chloride ethanol solution was transferred to a HDPE bottle (1 L). An aliquot was determined to be 11% hydrogen chloride by titration using aqueous potassium hydroxide (0.1N)

In a 5 L three neck round bottom flask ethyl alcohol (2528.0 g) was charged directly into the flask using a funnel. The hydrogen chloride in ethyl alcohol (359.7 g) was weighed directly into the ethyl alcohol in the flask. The 5 L three neck round bottom flask was placed on a support ring and equipped with an air driven stirrer, stirring shaft, bearing and TEFLON® blade and the contents of the flask were mixed for 5 minutes.

400.4 grams paroxetine acetate was added portion wise to the mixing solution using an addition funnel. To complete the transfer, ethyl alcohol was used to dissolve any material remaining on the funnel and neck of round bottom flask. The mixing was continued for 13 minutes.

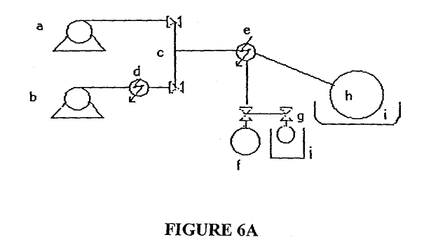

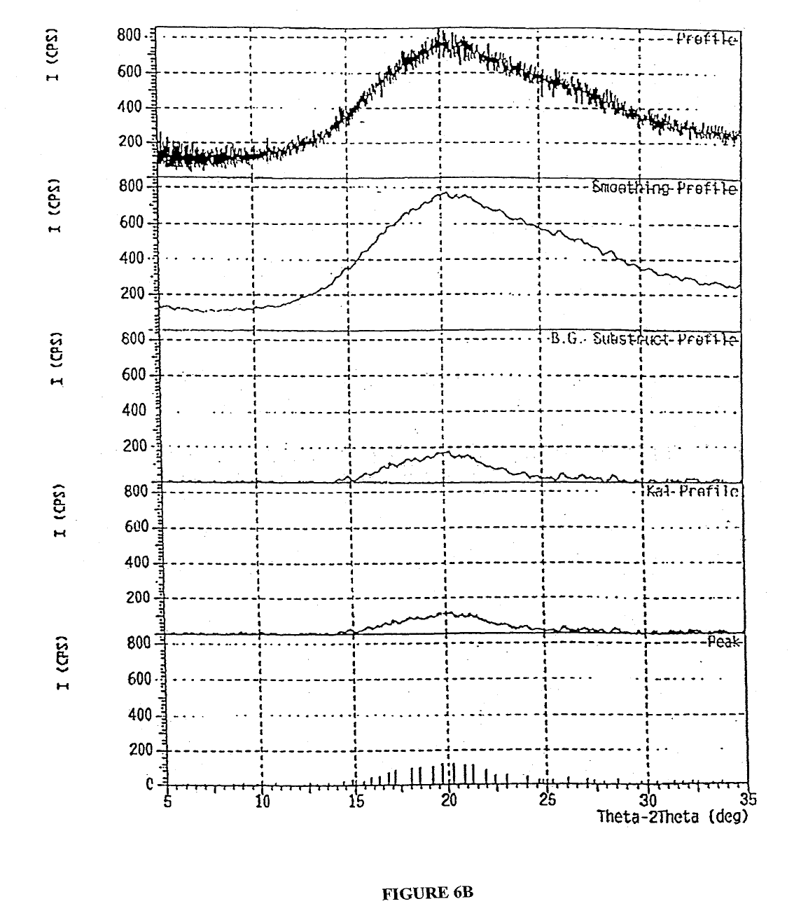

The Buchi rotory evaporator (model R-153) was then prepared for operation with changes made according to FIG. 6. The 20 L recovery flask was weighed and the weight was recorded. Two Teflone feed tubes were attached such that the inside tube led up to the neck of the 20 L flask and the other was attached outside to the feed stopcock. The 20 L dedicated recovery flask was attached. The rotary evaporator was configured with a 3-way TFE vacuum valve in such a way that the Buchi vacuum pump (model V-512) equipped with Buchi vacuum controller B-712 was directly connected to the valve and the Welch vacuum pump (model Dirrectorr 8910) was connected through a vacuum trap placed in a wide mouth Dewar flask, as shown in FIG. 6A. All the tubes, valves and trap between Welch pump and rotavapor had an inner diameter of 10 mm.

The Buchi rotary evaporator had two ports for the receiver flasks. A 10 L receiver flask was attached to the port closest to the condenser and the other 10 L receiver was replaced by a custom made 500 ml receiver flask and attached to the other port. Both valves to receiver from condenser were kept open. The distilled solvent collected first in the 10 L receiver as long as it was open. The 500 ml receiver was chilled in a dewer with liquid nitrogen while the 10 L receiver was at room temperature.

The vacuum trap before the Welch vacuum pump was chilled with liquid nitrogen. The rotary evaporator water bath was set to 60° C., the flask rotation speed at 65 RPM and the coolant circulated through the Buchi condenser. A Lauda chiller (WK 3200) was used to circulate coolant, and the chiller set point was adjusted to −2.0° C. The integrity of the systems to hold a vacuum was checked using the Buchi and Welch vacuum pumps at 2 Torr. The Welch vacuum pump lowered pressure in the system from 75 Torr to 2 Torr in less than 90 seconds. A test of the system showed that it could hold a vacuum at 2 Torr for 10 minutes. After testing the system the valve connected to the Welch vacuum pump on the 3-way TFE vacuum valve was closed.

The Buchi pump was set at 200 Torr and the paroxetine hydrochloride in ethanol solution was rapidly transferred using the feed tube attached to the rotory evaporator by suction into the rotating 20 L flask (2.5 minutes). To complete the transfer, the glassware and the suction tube were rinsed twice with ethyl alcohol (50 ml). The vacuum pumps were turned off and the flask was rotated at 60° C. for 60 minutes at atmospheric pressure.

The Buchi vacuum was then turned on and the set point of Buchi pump was lowered to 60 Torr. As the pressure dropped, the ethyl alcohol vapors began to condense and collect in the 10 L receiver. After 3.3 L of ethyl alcohol had been distilled only a clear viscous pale to colorless oil remained. The rotation speed was increased to 145 RPM. The Welch vacuum pump was then turned on.

The set point of the Buchi vacuum pump was changed to 1 Torr (1.3 mbar) and the valve to the 10 L receiver port was closed and the solvent was observed to ensure that it flowed to the 500 ml receiver.

The 3-way TFE vacuum valve was opened such that the high vacuum pump (Welch) and the Buchi vacuum pump were both working to evacuate the rotory evaporator. Significant foaming occurred at 14 Torr and was complete at 2 Torr. Pressure of 2 Torr in the system was achieved within 15 minutes after engaging the high vacuum pump. The bulk foam formed on the rotory evaporator continued to be dried at less than 3 Torr and 60±1° C. bath temperature for 60 minutes after the formation of foam was completed.

The vacuum pumps were turned off and the vacuum was released by purging the system with nitrogen. An oven was then preheated at 40° C. The 20 L flask with 387.6 g product was removed from the rotory evaporator. The foam paroxetine hydrochloride was scraped and blended to a fine powder inside the recovery flask using a polypropylene scoop. The flask was immediately covered to protect the contents from moisture.

The paroxetine hydrochloride amorphous (365.6g) was placed in glass dishes (150 mm×75 mm) and inserted in an oven at 40° C., and evacuated using house vacuum and purged with nitrogen three (3) times. Finally, a house vacuum of 25-29 inches Hg was applied for 16.5 hours to dry the bulk paroxetine hydrochloride.

The vacuum was released using nitrogen and the oven was maintained under a slight positive nitrogen pressure. The dishes containing the dried product were removed and weighed. The dried paroxetine hydrochloride yielded 355.7 g, a 9.9 g (2.7%) weight loss on drying.

The dried paroxetine hydrochloride, amorphous, was packaged after removing it from the vacuum oven in a zip press polyethylene bag (16″×20″). The bag was placed in another zip press bag (16″×20″) and over-packed with desiccant.

The 355.7 g of paroxetine hydrochloride was prepared as an amorphous off-white powder having an optical rotation of [α]²³_{D}=−87.9° at 0.5% in ethanol, and having a purity of 101.3% as determined by an HPLC assay as described above. A melting point determination showed that the paroxetine hydrochloride started to shrink at 73° C. and started transitioning to a clear gel at 86° to 89° C., where melting completed at 110° C. and at 130° C., at which temperatures no further movement was observed. Powder X-ray diffraction spectra for the amorphous solid are set forth in FIG. 6B. The dried solid was found to contain about 0.8 wt % water and about 0.6 wt % ethanol. Analysis by HPLC showed absence of related substances and impurities.

### EXAMPLE 16

### Synthesis of (−)-trans-4R-(4′-fluorophenyl -3S-[(3′,4′-methylenedioxyphenyl)oxymethyl]piperidine hydrochloride, amorphous (amorphous paroxetine hydrochloride) from paroxetine acetate

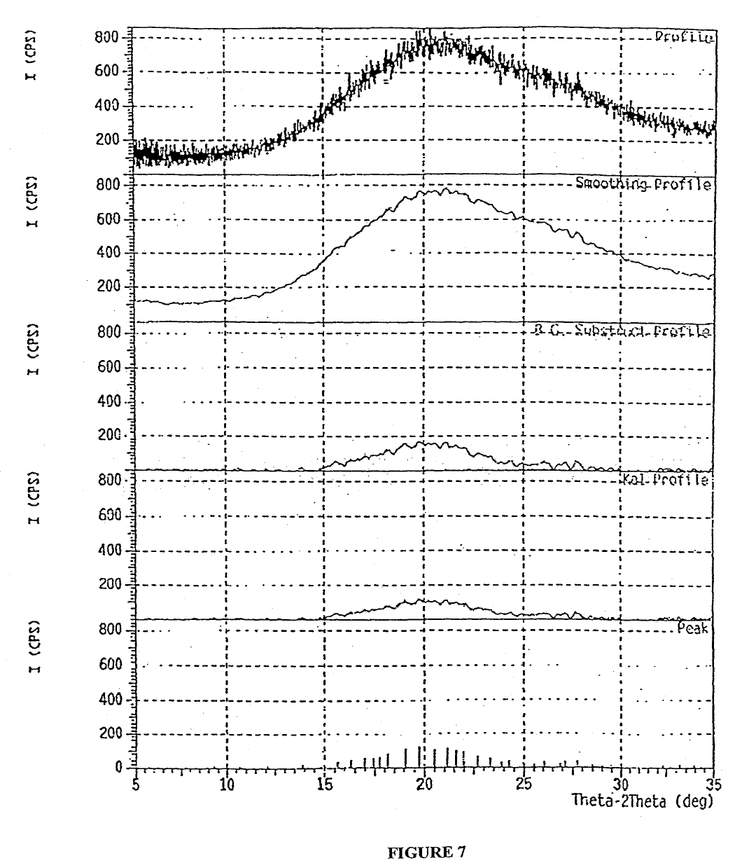

40.0 g (102.7 mmoles) paroxetine acetate was added slowly, with stirring, to a mixture of anhydrous methanol (300 ml) and 25% solution of hydrogen chloride in methanol (15.6 g, 3.9 g hydrogen chloride). The resultant clear solution was evaporated under reduced pressure using house vacuum (60 to 80 Torr) in a rotary evaporator at a 40° C. water bath temperature to a colorless oil. It was immediately subjected to a high vacuum (0.5 to 1 Torr) using a Welch pump at a 45° C. bath temperature whereupon foam formed. The foam was continued to be dried on a rotary evaporator for an additional 60 minutes at the same conditions. It was carefully removed from the flask to yield a 36.5 g white powder. Drying in a vacuum oven at 40° C. and 27-28 inch Hg pressure for 10 hours provided 36.0 g amorphous paroxetine hydrochloride as a white free flowing powder as proven by its X-ray powder diffraction analysis (FIG. 7). The amorphous solid had a melting point between 75° C. to 88° C. (75° C. starts shrinking, 88° C. becomes transparent gel, 110° C. to 122° C. gel starts moving and settles) and an optical rotation of [α]²⁵_{D}=−91 in 1% in ethanol.

What is claimed is:

1. A process for preparing amorphous paroxetine hydrochloride, which comprises mixing a carboxylic acid salt of paroxetine with hydrogen chloride and isolating the amorphous paroxetine hydrochloride.

2. The process of claim 1, wherein about 1.0 to about 4.0 mole equivalents of the hydrochloric acid to the paroxetine salt is utilized.

3. The process of claim 1, wherein the carboxylic acid salt of paroxetine and the hydrogen chloride are mixed in a solvent.

4. The process of claim 3, wherein the solvent is a low boiling linear alkanol or ester thereof.

5. The process of claim 4, wherein the solvent is ethanol.

6. The process of claim 1, wherein the carboxylic acid salt of paroxetine is selected from the group consisting of paroxetine formate, paroxetine acetate, paroxetine propionate, and paroxetine trifluoroacetate.

7. The process of claim 6, wherein the carboxylic acid salt of paroxetine is paroxetine acetate.

8. The process of claim 3, wherein the solvent is an alkanol and wherein the process further comprises conversion of a free carboxylic acid to an ester corresponding to the solvent alkanol.

9. The process of claim 8, wherein the free carboxylic acid is converted to the ester in the presence of excess hydrochloric acid in an amount of 0.01 to 0.1 mole equivalents of the paroxetine salt.

10. The process of claim 8, wherein the amount of solvent utilized maintains the solubility of the paroxetine hydrochloride in solution at 14° C. to 75° C.

11. The process of claim 3, further comprising removal of all or a portion of the solvent.

12. The process of claim 11, wherein the solvent is removed by evaporation.

13. The process of claim 12, wherein the evaporation is carried out at an elevated temperature, reduced pressure, or elevated temperature and reduced pressure.

14. The process of claim 12, wherein the evaporation is carried out by a rotary evaporator.

15. The process of claim 12, wherein the evaporation is carried out at a temperature of 35° C. to 75° C. and a pressure of 300 Torr to 10 Torr.

16. The process of claim 12, wherein the evaporation yields the paroxetine hydrochloride in the form of an oil.

17. The process of claim 16, wherein the oil is formed and maintained at about 60 Torr to 10 Torr and at 35 to 75° C.

18. The process of claim 16, which further comprises removing all or part of the solvent from the oil to yield the amorphous paroxetine hydrochloride in the form of a foam.

19. The process of claim 18, wherein the solvent is removed at a temperature of 35° C. to 75° C. and a pressure of 60 Torr to 0.01 Torr.

20. The process of claim 18, wherein the temperature is about 60° C. and the pressure is about 30 to about 0.1 Torr.

21. The process of claim 18, wherein a rotary evaporator is used for the production of foam.

22. The process of claim 18, which further comprises drying the foam to produce the amorphous paroxetine hydrochloride as a free flowing solid.

23. The process of claim 22, wherein the drying is carried out in an oven.

24. The process of claim 23, wherein the oven is a vacuum oven.

25. The process of claim 1, wherein the paroxetine hydrochloride amorphous is substantially pure.

26. The process of claim 1, wherein the paroxetine hydrochloride comprises less than 10% wt/wt of an alkanol.

27. The process of claim 26, wherein the alkanol is ethanol.

28. The process of claim 1, wherein the paroxetine hydrochloride comprises about 0.01% to about 3% wt/wt of water.

29. A process for purifying paroxetine free base, which comprises
mixing paroxetine free base in a water immiscible organic solvent with water and an acid to yield a water soluble paroxetine salt in water,
removing the organic solvent,
mixing the water soluble paroxetine salt in water with a water immiscible organic solvent and an aqueous base to yield purified paroxetine free base in the organic solvent; and
separating the organic solvent having purified paroxetine free base from the mixture.

30. The process of claim 29, wherein the acid is a carboxylic acid and the water soluble paroxetine salt is a carboxylic acid salt of paroxetine.

31. The process of claim 30, wherein the carboxylic acid is acetic acid or formic acid and the carboxylic acid salt is paroxetine acetate or paroxetine formate.

32. The process of claim 29, wherein the water immiscible organic solvent is toluene.

33. A process for determining the content of paroxetine free base in a solution, which comprises
removing an aliquot from the solution;
evaporating solvent from the aliquot to yield paroxetine free base as an oil that is substantially free of solvent; and
calculating the amount of free base in the solution from the amount of paroxetine free base obtained as the oil in the aliquot.

34. A process for preparing a solid paroxetine salt, which comprises
mixing paroxetine free base in a water immiscible organic solvent with water and a first acid to yield a water soluble paroxetine salt in water,
removing the organic solvent,
mixing the water soluble paroxetine salt in water with a water immiscible organic solvent and an aqueous base to yield purified paroxetine free base in the water immiscible solvent;
separating the organic solvent having purified paroxetine free base from the mixture; and
mixing the purified paroxetine free base with a second acid in a solvent and isolating the solid paroxetine salt.

35. The process of claim 34, wherein the solvent is toluene.

36. The process of claim 34, wherein the second acid is a carboxylic acid.

37. The process of claim 36, wherein the carboxylic acid is selected from the group consisting of formic acid, acetic acid, propionic acid, and trifluoroacetic acid.

38. The process of claim 36, wherein the ratio of the volume of purified free base in solvent to the weight of purified free base is about 7.5 to about 10 milliliters per gram and the amount of the carboxylic acid is about 1 mole equivalent to paroxetine free base in the solution.

39. The process of claim 34, wherein the acid is selected from the group consisting of sulfuric acid, phosphoric acid, and carbonic acid.

40. The process of claim 39, wherein the acid is sulfuric acid, the solvent is an alcohol, and the molar ratio of sulfuric acid to paroxetine free base is about 0.5.

41. The process of claim 39, wherein the acid is phosphoric acid and the molar ratio of phosphoric acid to paroxetine free base is about 1.0.

42. A composition of matter comprising paroxetine acetate, characterized as having IR absorption bands at 653, 785, 795, 841, 935, 985, 1036, 1105, 1141, 1187, 1219, 1241, 1269, 1382, 1421, 1471, 1489, 1503, 1512, 1629, 1654, 2888, 2934, 3056, 3083±4 cm⁻¹, and having X-ray powder diffraction peaks at 19.1, 20.4 and 21.5 (2 Theta degree).

43. A composition of matter comprising paroxetine formate, characterized as having IR absorption bands at 762, 785, 837, 934, 1040, 1138, 1188, 1218, 1245, 1269, 1343, 1365, 1382, 1471, 1489, 1505, 1571, 1634, 1689, 1784, 1891, 3045±4 cm⁻¹, and having X-ray powder diffraction peaks at 19.4, 20.1 and 23.1 (2 Theta degree).

44. A composition of matter comprising paroxetine trifluoroacetate.

45. The composition of matter of claim 44, wherein the paroxetine trifluoroacetate has a melting point that is less than or equal to 117° C.

46. A composition of matter comprising paroxetine carbonate or paroxetine hydrogen carbonate.

47. A composition of matter comprising paroxetine sulfate or paroxetine hydrogen sulfate.

48. The composition of claim 47, wherein the composition of matter comprises paroxetine sulfate having a melting point that is less than or equal to 245° C.

49. A composition of matter comprising paroxetine phosphate, paroxetine hydrogen phosphate, or paroxetine dihydrogen phosphate.

50. The composition of claim 51, wherein the composition comprises paroxetine dihydrogen phosphate having a melting point that is less than or equal to 208° C.

51. A process for preparing a paroxetine salt, comprising
contacting paroxetine free base in an organic solvent with an acid selected from the group consisting of sulfuric acid, phosphoric acid, propionic acid, trifluoroacetic acid, and carbonic acid; and
isolating the paroxetine salt.
